(19) United States
(12) Patent Application Publication
(10) Pub. No.: US 2018/0110795 A1
(43) Pub. Date: Apr. 26, 2018

(54) COMPOSITIONS AND METHODS FOR DETECTING AND TREATING PERIODONTAL DISEASE

(71) Applicant: THE FORSYTH INSTITUTE, CAMBRIDGE, MA (US)

(72) Inventor: JORGE FRIAS-LOPEZ, CAMBRIDGE, MA (US)

(73) Assignee: THE FORSYTH INSTITUTE, CAMBRIDGE, MA (US)

(21) Appl. No.: 15/568,929

(22) PCT Filed: Apr. 22, 2016
(86) PCT No.: PCT/US16/29010

§ 371 Date: Oct. 24, 2017

Related U.S. Application Data
(60) Provisional application No. 62/152,579, filed on Apr. 24, 2015.

(51) Int. Cl. A61K 31/7076; A61K 31/4025; A61K 31/4015; A61K 39/40; A61K 9/68; A61Q 11/00; A61P 43/00; G01N 33/50; G01N 33/68; G01N 33/82; G01N 33/84

(52) U.S. Cl. CPC A61K 31/7076; A61K 31/4025; A61K 31/4015; A61K 39/40; A61K 9/0058; A61Q 11/00; G01N 2800/7066; G01N 33/5023; G01N 33/6893; G01N 33/82; G01N 33/84; G01N 2800/18; A61P 43/00

(57) ABSTRACT

The invention features compositions comprising inhibitors of cobalamin biosynthesis, methods of detecting periodontal disease from a subject's subgingival plaque using a panel of biomarkers, and methods of treating periodontal disease using oral formulations.

### CROSS REFERENCE TO RELATED APPLICATIONS

This application claims benefit of U.S. Provisional Application Ser. No. 62/152,579, filed Apr. 24, 2015, the contents of which are incorporated herein by reference.

### STATEMENT OF RIGHTS TO INVENTIONS MADE UNDER FEDERALLY SPONSORED RESEARCH

This work was supported by the following grants from the National Institute of Dental and Craniofacial Research of the National Institutes of Health (NIDCR/NIH), Grant Nos: DE021553 and DE021127. The government has certain rights in the invention.

### BACKGROUND OF THE INVENTION

Among the oral conditions caused by a dysbiotic microbial community, periodontitis is the sixth most prevalent health condition in the world affecting 743 million people worldwide and occurs in moderate form in 30% to 50% of American adults and in severe form in 10% of the population and it is responsible for half of all tooth loss in adults. In addition, recent studies have indicated that periodontal diseases can influence the risk for certain systemic conditions such as cardiovascular diseases, diabetes, respiratory diseases, and can affect reproductive outcome. Moreover, periodontal therapy may improve health outcomes in different systemic conditions, such as type 2 diabetes, coronary artery disease, cerebral vascular disease, rheumatoid arthritis and pregnancy. Accordingly, methods for treating and detecting periodontal diseases early are urgently required.

### SUMMARY OF THE INVENTION

As described below, the present invention features compositions comprising inhibitors of cobalamin biosynthesis, methods of detecting periodontal disease from a subject's subgingival plaque using a panel of biomarkers, and methods of treating periodontal disease using oral formulations.

In one aspect, the invention provides an oral formulation containing a cobalamin synthesis inhibitor to treat or prevent periodontal disease and related disorders.

In another aspect, the invention provides a method for identifying a subject having or at risk of developing periodontal disease involving detecting altered expression of a gene associated with cobalamin synthesis, urea metabolism, citrate transport, iron ion transport, potassium ion transport, amino-acid transport, isoprenoid biosynthesis and ciliary and flagellar motility in a bacteria associated with periodontal disease relative to a reference. In certain embodiments, the expression of a gene associated with cobalamin synthesis is increased, relative to a reference. In certain embodiments, the expression of a gene associated with potassium ion transport is decreased, relative to a reference.

In yet another aspect, the invention provides a method for identifying a subject having or at risk of developing periodontal disease involving detecting an increase in expression of a polypeptide or gene encoding a polypeptide associated with cobalamin synthesis or a decrease in expression of a polypeptide or gene encoding a polypeptide involved in potassium ion transport in a bacteria associated with periodontal disease relative to a reference.

In still another aspect, the invention provides a method of treating or preventing periodontal disease by administering an oral formulation involving a cobalamin synthesis inhibitor in a subject identified by an increase in expression of a polypeptide or gene involved in cobalamin synthesis or a decrease in expression of a polypeptide or gene encoding a polypeptide involved in potassium ion transport in a bacteria associated with periodontal disease relative to a reference.

In one aspect, the invention provides a kit for detecting periodontitis in a subject, the kit containing a panel of capture molecules that detect an alteration in cobalamin synthesis and/or potassium ion transport.

In another aspect, the invention provides a kit for treating or preventing periodontitis, the kit containing an effective amount of a cobalamin synthesis inhibitor.

In various embodiments of any of the previous aspects or any other aspect of the invention delineated herein, the cobalamin synthesis inhibitor is one or more of 19-bromo-1-hydroxymethylbilane, N(D)-methyl-1-formylbilane, N-ethylmaleimide, adenylyl-imidodiphosphate, adenylyl(b,g-methylene)-diphosphonate, ADP, protonpump inhibitor (PPi), divalent metal ions, S-adenosyl-L-homocysteine, tripolyphosphate/sodium triphosphate, and hydrogenobyrinic acid a,c-diamide.

In various embodiments of any of the previous aspects or any other aspect of the invention delineated herein, the cobalamin synthesis inhibitor reduces the level, activity, or expression of one or more cobalamin synthesis nucleic acids or polypeptides relative to a reference.

In various embodiments of any of the previous aspects or any other aspect of the invention delineated herein, the polypeptide is one or more of Delta-aminolevulinic acid dehydratase, Porphobilinogen deaminase, Uroporphyrinogen II synthase, Siroheme synthase, Precorrin-2 C20-methyltransferase, Precorrin-3B synthase, Precorrin-3B C17-methyltransferase, Precorrin-4 C11-methyltransferase, Precorrin-6A synthase, Precorrin-6X reductase, Precorrin-6Y C5,15-methyltransferase, Precorrin-8X methylmutase, Cobyrinic acid a,c-diamide synthase, Cobaltochelatase, Adenosylcobinamide-GDP ribazoletransferase, Nicotinate-nucleotide-dimethylbenzimidazole phosphoribosyltransferase, Cob(I)yrinic acid a,c-diamide adenosyltransferase, Cobyric acid synthase, Threonine-phosphate decarboxylase, Threonine-phosphate decarboxylase, Adenosylcobinamide kinase/Adenosylcobinamide-phosphate guanylyltransferase, Cobalamin-5-phosphate synthase, and Adenosylcobinamide kinase/Adenosylcobinamide-phosphate guanylyltransferase. In various embodiments, the cobalamin synthesis inhibitor is one or more of an inhibitory nucleic acid, polypeptide, enzyme, or siRNA.

In various embodiments of any of the previous aspects or any other aspect of the invention delineated herein, the oral formulation contains a toothpaste, powder, liquid dentifrice, mouthwash, subgingival irrigation fluid, mouth spray, mouth rinse, topically applied solution, denture cleanser, mouth guard, chewable tablets, chewing gum, lozenge, paste, gel, ointment, mucoadhesive, bioerodable film, buccal wafers, chocolate pieces, bars or nougats or candy. In various embodiments the oral formulation contains a coated fiber. In various embodiments said coated fiber is floss. In various embodiments said coated fiber is toothbrush bristle.

In various embodiments of any of the previous aspects or any other aspect of the invention delineated herein, the oral formulation contains an interproximal dental brush.

In various embodiments of any of the previous aspects or any other aspect of the invention delineated herein, the bacteria is Prevotella nigrescens, Prevotella intermedia, Fusobacterium nucleatum subspecies nucleatum, Tannerella forsythia, or Porphyromonas gingivalis.

In various embodiments of any of the previous aspects or any other aspect of the invention delineated herein, the gene is one identified at Table 5.

Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “bacteria associated with periodontal disease” is meant any bacteria that functions in periodontal disease pathology. Exemplary bacteria include Prevotella nigrescens, Prevotella intermedia, Fusobacterium nucleatum subspecies nucleatum, Tannerella forsythia, or Porphyromonas gingivalis.

By “cobalamin synthesis inhibitor” is meant any agent, such as a molecule, nucleic acid, polynucleotide, protein, siRNA, enzyme or antibody that reduces or eliminates cobalamin synthesis. A cobalamin synthesis inhibitor specifically binds and/or reduces the activity or expression of a cobalamin synthesis pathway nucleic acid or protein. Examples of cobalamin inhibitors include, but are not limited to 19-bromo-1-hydroxymethylbilane, N(D)-methyl-1-formylbilane, N-ethylmaleimide, adenylyl-imidodiphosphate, adenylyl(b,g-methylene)-diphosphonate, ADP, protonpump inhibitor (PPᵢ), divalent metal ions, S-adenosyl-L-homocysteine, tripolyphosphate/sodium triphosphate, and hydrogenobyrinic acid a,c-diamide.

By “co-formulated” is meant any single pharmaceutical composition which contains two or more therapeutic or biologically active agents.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “degrades” is meant physically or chemically breaks down in whole or in part. Preferably, the degradation represents a physical reduction in the mass or structural integrity of a material (i.e., a film, adhesive, or composite) by at least about 10%, 25%, 50%, 75%, 80%, 85%, 90%, 95% or 100%.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include periodontal disease, gingivitis, mucosal membrane lesions, gum bleeds, plaque-induced inflammation, and bone or tooth loss.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat periodontal disease, gingivitis, mucosal membrane lesions, gum bleeds, plaque-induced inflammation, and bone or tooth loss in the oral cavity by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Markers of the invention include genes that are altered in connection with periodontal disease and related disorders, such genes include genes that function in the cobalamin synthesis pathway and/or those that encode potassium transporters.

As used herein, “obtaining” as in “obtaining an agent” includes synthesizing, purchasing, or otherwise acquiring the agent.

By “periodontitis” is meant one or more conditions associated with periodontal disease, gingivitis, bone and tooth loss, gum bleeds, mucosal membrane lesions and inflammations in the oral cavity.

By “pharmaceutical preparation” or “pharmaceutical composition” is meant any composition which contains at least one therapeutically or biologically active agent and is suitable for administration to a patient. For the purposes of this invention, pharmaceutical compositions suitable for delivering a therapeutic to oral cavity include, but are not limited to solutions and suspensions delivered either as an oral spray or rinse, pastes, gels, chewable tablets, sublingual, gingival, or buccal wafers and films, chewing gum, lozenges, toothpaste, powder, liquid dentifrice, mouthwash, subgingival irrigation fluid, topically applied solution, denture cleanser, mouth guard, gel, ointment, mucoadhesive, chocolate pieces, bars or nougats or candy and other compositions designed to be retained in the mouth for an extended period of time. Any of these formulations can be prepared by well known and accepted methods of art. See, for example, Remingtion: The Science and Practice of Pharmacy, .sub.19th edition, (ed. AR Gennaro), Mack Publishing Co., Easton, Pa., 1995.

By “reduces” is meant a negative alteration of at least 10%, 25%, 50%, 75%, or 100%.

By “reference” is meant a standard or control condition. In certain embodiments, the reference is a non-diseased tissue or sample.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C show three circular graphs of statistical differences in metagenome composition. Metagenome hit counts were first normalized using genome abundance similarity correction (GASiC). Normalized counts were then analyzed using linear discriminant analysis effective size (LEfSe) with default parameters to identify significant differences at species level between the microbial communities compared.

FIG. 1A is a circular graph showing a comparison of baseline samples from active sites vs. periodontal disease progressing samples from active sites (i.e. samples collect at the visit when an increase in CAL≥2 mm was detected).

FIG. 1B is a circular graph showing a comparison of baseline samples from stable sites vs. follow-up samples from stable sites (i.e. collected 2 months after baseline).

FIG. 1C is a circular graph showing a comparison of baseline samples from active sites vs. baseline samples from stable sites.

FIGS. 2A and 2B show two circular graphs of statistical differences in metagenome composition. Metagenome hit counts were first normalized using genome abundance similarity correction (GASiC). Normalized counts were then analyzed using linear discriminant analysis effective size (LEfSe) with default parameters to identify significant differences at species level between the microbial communities compared.

FIG. 2A is a circular graph showing a comparison of non-progressing site baselines to healthy sites of healthy patients.

FIG. 2B is a circular graph showing a comparison of periodontal disease progressing site baselines to healthy sites of healthy patients.

FIGS. 3A-3C show three circular graphs of statistical differences in metatranscriptome normalized composition. Metatranscriptome hits were first normalized by the relative frequency of species obtained in the metagenomic analysis using genome abundance similarity correction (GASiC). Normalized counts were then analyzed using linear discriminant analysis effective size (LEfSe) with default parameters to identify significant differences in activity at the species level.

FIG. 3A is a circular graph showing a comparison from active sites vs. periodontal disease progressing samples from active sites.

FIG. 3B is a circular graph showing a comparison of baseline samples from stable sites vs. follow-up samples from stable sites (i.e. collected 2 months after baseline).

FIG. 3C is a circular graph showing a comparison of baseline samples from active sites vs. baseline samples from stable sites.

FIG. 4 is a circular graph showing statistical differences in normalized metatranscriptome composition comparing non-progressing site baselines to. healthy sites of healthy patients. Metagenome hit counts were first normalized using genome abundance similarity correction (GASiC). Metatranscriptome normalized counts were then analyzed using linear discriminant analysis effective size (LEfSe) with default parameters to identify significant differences in activity at species level between the microbial communities compared.

FIG. 5 is a circular graph showing statistical differences in normalized metatranscriptome composition comparing periodontal disease progressing site baselines to healthy sites of healthy patients. Metagenome hit counts were first normalized using genome abundance similarity correction (GASiC). Metatranscriptome normalized counts were then analyzed using linear discriminant analysis effective size (LEfSe) with default parameters to identify significant differences in activity at species level between the microbial communities compared.

FIGS. 6A and 6B show scatter plots of Gene Ontology (GO) enrichment analysis comparing baseline in active sites to periodontal disease progression profiles in the same sites. Enriched terms obtained using ‘GOseq’ were summarized and visualized as a scatter plot using reduce and visual Gene Ontology (REVIGO) method.

FIG. 6A is a scatter plot showing summarized GO terms related to biological processes at baseline.

FIG. 6B is a scatter plot showing summarized GO terms related to biological processes in periodontal disease progression. Circle size is proportional to the frequency of the GO term, while color indicates the log 10 p value (red higher, blue lower).

FIG. 7 is a Venn diagram showing overlapping differentially expressed genes comparing baseline and periodontal disease progression with and without normalization. The expression hit counts against species frequencies estimated were normalized using genome abundance similarity correction (GASiC). Venn diagram was obtained using the Venny webpage tool, a computational genomics service offering a Venn's diagrams drawing tool for comparing up to four lists of elements.

FIGS. 8A and 8B show diagrams of Gene Ontology (GO) terms associated with changes in gene expression profiles in major periodontal pathogens members of the red complex during periodontal disease progression. GO terms were assigned to differentially expressed genes in periodontal disease progression and summarized using reduce and visual Gene Ontology (REVIGO) method.

FIG. 8A is a diagram showing GO terms associated with up-regulated genes in active sites.

FIG. 8B is a diagram showing GO terms associated with down-regulated genes in active sites.

FIGS. 9A and 9B show scatter plots of Gene Ontology (GO) enrichment analysis comparison of baselines from periodontal disease progressing and non-progressing sites. Enriched terms obtained using ‘GOseq’ were summarized and visualized as a scatter plot using reduce and visual Gene Ontology (REVIGO) method.

FIG. 9A is a scatter plot showing summarized GO terms related to biological processes in baselines of periodontal disease progressing sites.

FIG. 9B is a scatter plot showing summarized GO terms related to biological processes in baselines of non-progressing sites. Circle size is proportional to the frequency of the GO term, while color indicates the log 10 p value (red higher, blue lower).

FIGS. 10A-10D show four scatter plots of Gene Ontology (GO) enrichment analysis comparing healthy sites from healthy individuals and baselines in active sites and inactive sites. Enriched terms obtained using ‘GOseq’ were summarized and visualized as a scatter plot using reduce and visual Gene Ontology (REVIGO) method.

FIG. 10A shows a scatter plot of summarized GO terms related to biological processes in inactive baselines.

FIG. 10B shows a scatter plot of summarized GO terms related to biological processes in health when compared with inactive baselines.

FIG. 10C shows a scatter plot of summarized GO terms related to biological processes in active baselines.

FIG. 10D shows a scatter plot of summarized GO terms related to biological processes in health when compared with active baselines. Circle size is proportional to the frequency of the GO term, while color indicates the log 10 p value (red higher, blue lower). q-value=0.9.

FIGS. 11A and 11B show two diagrams of Gene Ontology (GO) terms associated with changes in gene expression profiles in major periodontal pathogens members of the red complex when comparing baselines of active and inactive sites. GO terms were assigned to differentially expressed genes in periodontal disease progression and summarized using reduce and visual Gene Ontology (REVIGO) method.

FIG. 11A is a diagram showing GO terms associated with up-regulated genes in active sites baselines.

FIG. 11B is a diagram showing GO terms associated with down-regulated genes in active sites baselines.

FIGS. 12A and 12B show two diagrams of Gene Ontology (GO) terms associated with changes in gene expression profiles in members of the orange complex when comparing baselines of active and inactive sites. GO terms were assigned to differentially expressed genes in periodontal disease progression and summarized using reduce and visual Gene Ontology (REVIGO) method.

FIG. 12A is a diagram showing GO terms associated with up-regulated genes in active sites baselines.

FIG. 12B is a diagram showing GO terms associated with down-regulated genes in active sites baselines.

FIGS. 13A and 13B are tables showing ranked species by the number of up-regulated putative virulence factors in the metatranscriptome. Putative virulence factors were identified by alignment of the protein sequences from the different genomes against the Virulence Factors Database (VFDB) as described in the methods section. Numbers in the graph refer to absolute number of hits for the different species for the putative virulence factors identified. In red are the members of the red complex. In orange are members of the orange complex.

FIG. 13A is a table showing a comparison of baseline to periodontal disease progressing.

FIG. 13B is a table showing a comparison of baseline non-progressing to. baseline progressing.

FIGS. 14A-14C show three diagrams of Gene Ontology (GO) terms associated with changes in gene expression of putative virulence factors in the oral community during periodontital disease progression. GO terms were assigned to differentially expressed putative virulence factors in periodontal disease progressing periodontal disease progression and summarized using reduce and visual Gene Ontology (REVIGO) method.

FIG. 14A is a diagram showing GO terms enrichment analysis of virulence factors in the whole community.

FIG. 14B is a diagram showing GO terms associated with up-regulated virulence factors in the red complex.

FIG. 14C is a diagram showing GO terms associated with up-regulated virulence factors in the orange complex.

FIGS. 15A and 15B show two diagrams of Gene Ontology (GO) terms enrichment analysis of virulence factors comparing baselines. GO terms enrichment was performed using GOseq and summarized using reduce and visual Gene Ontology (REVIGO).

FIG. 15A is a diagram showing GO terms over-represented in periodontal disease progressing sites baselines.

FIG. 15B is a diagram showing GO terms over-represented in non-progressing sites baselines.

FIG. 16 shows a diagram of Gene Ontology (GO) terms enrichment analysis of virulence factors in the orange complex comparing baselines. GO terms enrichment was performed using GOseq and summarized using reduce and visual Gene Ontology (REVIGO). FIG. 16 is a diagram showing GO terms over-represented in baselines of periodontal disease progressing sites.

FIGS. 17A and 17B show Correlation Circle plots of sparse Partial Least Square (sPLS) analysis. Correlation Circle plots were obtained to assess correlation of the evolution of bleeding on probing (BOP), increase in pocket depth (ΔPD) and increase in clinical attachment level (ΔCAL).

FIG. 17A is a Correlation Circle plot showing a 3D representation of gene expression associations with evolution of clinical traits (components 1 to 3).

FIG. 17B is a Correlation Circle plot showing gene expression associations with evolution of clinical traits of the 2 first components.

FIGS. 18A and 18B show two diagrams of Relevance Networks for the association of clinical parameters and active bacterial species. Relevance Networks were obtained for the first three sparse Partial Least Square (sPLS) dimensions. A threshold of r=0.95 was used to select for association between periodontal disease progression of clinical parameters (ΔPD increase in pocket depth, ΔCAL increase in clinical attachment level) and gene expression profiles. GO terms were assigned to genes whose pattern of expression was significantly associated with the clinical parameters measured. GO terms were summarized using reduce and visual Gene Ontology (REVIGO) method.

FIG. 18A shows a diagram of GO terms associated with ΔPD.

FIG. 18B shows a diagram of GO terms associated with ΔCAL.

FIG. 19 show a bargraph depicting percentage of hits corresponding to viral sequences. Sequences were aligned against a database containing all viral sequences in NCBI. Bars represent the percentage of hits that corresponded to viral sequences.

FIG. 20 is a circular graph showing statistical differences in viral compositions of transcripts. Hit counts were analyzed using linear discriminant analysis effective size (LEfSe) with default parameters, to identify significant differences at species level between the microbial communities compared. Comparison periodontal disease progressing site baselines to end point of the same sites.

FIG. 21 includes six micrographs showing plaque biofilm in the presence or absence of sodium tripolyphosphate (TPP) at different concentrations, which is an inhibitor of Cob(I)alamin adenosyltransferase.

FIG. 22 includes a PCA analysis showing the effect of TPP on microbial communities where the presence of 500 oral species was assessed by 16SrRNA analysis.

### DETAILED DESCRIPTION OF THE INVENTION

The invention features compositions comprising inhibitors of cobalamin biosynthesis, methods of detecting periodontal disease from a subject's subgingival plaque using a panel of biomarkers, and methods of treating periodontal disease using oral formulations.

The invention is based, at least in part, on the discovery that the expression of genes that function in the cobalamin synthesis pathway and the potassium transporter are altered in bacteria that are involved in periodontal pathology. Accordingly, the invention provides cobalamin synthesis inhibitors, and methods of identifying subjects having or having the propensity to develop periodontal disease and associated conditions.

### Periodontal Disease

Periodontitis is a polymicrobial biofilm-induced inflammatory disease that affects 743 million people worldwide. The current model to explain periodontitis progression proposes that changes in the relative abundance of members of the oral microbiome lead to dysbiosis in the host-microbiome crosstalk and from there to inflammation and bone loss. Using combined metagenome/metatranscriptome analysis of the oral community in active and non-progressing sites during periodontitis progression the molecular activities of active and non-progressing sites were characterized.

Much has been learned about the diversity and distribution of oral associated microbial communities, but still little is known about the biology of the microbiome, how it interacts with the host, and how the host responds to its resident microbiota. The oral cavity offers a unique opportunity to study how microbial communities have an influence on the health status of their human host. Imbalances of the oral microbiota, also referred to as oral microbial dysbiosis, lead to a series of different oral diseases. These dysbiotic microbial communities exhibit synergistic interactions for enhanced protection from host defenses, nutrient acquisition, and persistence in an inflammatory environment.

Periodontitis is an oral polymicrobial disease caused by the coordinated action of a complex microbial community, which results in inflammation and destruction of the periodontium in susceptible hosts. Using checkerboard DNA-DNA hybridization technique, periodontitis-associated taxa have been cataloged into groups or complexes, representing bacterial consortia that appear to occur together and that are associated with various stages of disease (Socransky et al., Clin Periodontol. 25:134-44, 1998). The ‘red complex,’ which appears later in biofilm development, comprises three species that are considered the major periodontal pathogens: Porphyromonas gingivalis, Treponema denticola, and Tannerella forsythia. Another important group of organisms that has been associated with chronic periodontitis is the orange complex constituted by: Fusobacterium nucleatum, Prevotella intermedia, Prevotella nigrescens, Parvimonas micro, Streptococcus constellatus, Eubacterium nodatum, Campylobacter showae, Campylobacter gracilis and Campylobacter rectus. Similarly to the red complex, all species in the orange complex showed a significant association with increasing pocket depth (Socransky et al., Clin Periodontol. 25:134-44, 1998, Socransky et al., Periodontol 2000. 38:135-87, 2005) and reciprocal interactions between both have been proposed (Socransky et al., Periodontol 2000. 38:135-87, 2005). In more recent studies using 454 pyrosequencing to characterize healthy and periodontitis microbial communities the overall picture of bacterial associations with health and disease agree with the initial descriptions of the different oral microbial complexes.

Periodontitis leads to severe gingivitis and can cause mucosal membrane lesions and inflammations, gum bleeds, and tooth and bone loss and can be highly painful. While there are different causes for the disease, bacteria is the most common. Periodontitis is mostly a chronic disease requiring ongoing treatment, in some cases for months or even years. One of the questions to be answered regarding the pathogenesis of periodontitis is why in some cases teeth with clinical symptoms of periodontitis progress leading to tooth loss (if untreated) and in some other cases the progression of the disease stops despite lack of treatment. There have been a large number of attempts to identify reliable markers that would distinguish between active and non-progressing sites. Among those there are genetic markers, protein activity, cytokines, bacterial and clinical. However, none of these associations explain why periodontitis progression occurs.

Current models of periodontal disease progression posit that tissue destruction progresses through periods of acute exacerbations (activity) followed by periods of remission. It has been postulated that changes in the composition of subgingival biofilms could explain these periods of disease activity. In fact, a few papers have found differences in the levels of subgingival species when comparing periodontal disease progressing and non-progressing sites using cultural techniques and molecular approaches such as real-time PCR. However, these studies also demonstrated considerable overlap in the composition of the microbial communities associated with active and non-progressing lesions, indicating that the difference in the periodontal status of the sites could not be explained solely by the reported differences in the subgingival microbial composition.

Cataloging the activities of each bacterial species in a community may provide more insight into pathogenesis than simple enumeration of that community gene content. This is because the community functions as a system, and it is the activities and interactions of the system that control the fate of the microbiome.

The goal of the present study was to characterize in situ gene expression patterns of the whole oral microbiome during periodontitis progression to identify early steps in dysbiosis that could answer the question of why only certain teeth progress to disease and why other do not.

### Diagnostics

Plaque obtained from subjects has altered levels of particular biomarkers. In particular, subjects are identified as having periodontitis (e.g., gingivitis, mucosal membrane lesions, plaque-induced inflammations, gum bleeds, bone or tooth loss in the oral cavity), or a propensity to develop such conditions by detecting an alteration in one or more of genes or proteins involved in cobalamin synthesis and potassium ion transport obtained from the subject relative to the level of such biomarkers in a reference. Alterations in the levels of such biomarkers (or any other marker delineated herein) are detected using standard methods. In another approach, diagnostic methods of the invention are used to assay the expression of genes or proteins involved in cobalamin synthesis and potassium ion transport in a biological sample relative to a reference (e.g., the level of such polypeptides present in a corresponding control sample). In one embodiment, the level of proteins involved cobalamin synthesis and potassium ion transport is detected using an antibody that specifically binds the polypeptide. Exemplary antibodies that specifically bind such polypeptides are described herein. Such antibodies are useful for the diagnosis of periodontitis (e.g., gingivitis, mucosal membrane lesions, plaque-induced inflammations, gum bleeds, bone or tooth loss in the oral cavity), or a propensity to develop such conditions. Methods for measuring an antibody-marker complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of marker in a sample, where an increase in the level of the marker polypeptide is diagnostic of a patient having periodontitis (e.g., gingivitis, mucosal membrane lesions, plaque-induced inflammations, gum bleeds, bone or tooth loss in the oral cavity), or a propensity to develop such conditions.

In general, the measurement of a marker polypeptide in a subject sample is compared with a diagnostic amount present in a reference. A diagnostic amount distinguishes between periodontitis (e.g., gingivitis, mucosal membrane lesions, plaque-induced inflammations, gum bleeds, bone or tooth loss in the oral cavity), or a propensity to develop such conditions and the absence of such condition. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an marker polypeptide or nucleic acid molecule in the subject sample relative to a reference may be used to diagnose periodontitis (e.g., gingivitis, mucosal membrane lesions, plaque-induced inflammations, gum bleeds, bone or tooth loss in the oral cavity), or a propensity to develop such conditions. In one embodiment, the reference is the level of marker polypeptide present in a control sample obtained from a patient that does not have periodontitis (e.g., gingivitis, mucosal membrane lesions, plaque-induced inflammations, gum bleeds, bone or tooth loss in the oral cavity), or a propensity to develop such conditions. In another embodiment, the reference is a baseline level of marker present in a biologic sample derived from a patient prior to, during, or after treatment for a periodontitis (e.g., gingivitis, mucosal membrane lesions, plaque-induced inflammations, gum bleeds, bone or tooth loss in the oral cavity), or a propensity to develop such conditions. In yet another embodiment, the reference is a standardized curve.

In another approach, diagnostic methods of the invention are used to assay the expression of genes or proteins involved in cobalamin synthesis and potassium ion transport in a biological sample relative to a reference (e.g., the level of such polypeptides present in a corresponding control sample). In one embodiment, the level of genes or proteins involved in cobalamin synthesis and potassium ion transport is detected using an antibody that specifically binds the polypeptide. Exemplary antibodies that specifically bind such polypeptides are described herein. Such antibodies are useful for the diagnosis of periodontitis (e.g., gingivitis, mucosal membrane lesions, plaque-induced inflammations, gum bleeds, bone or tooth loss in the oral cavity), or a propensity to develop such conditions. Methods for measuring an antibody-marker complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of marker in a sample, where an increase in the level of the marker polypeptide is diagnostic of periodontitis (e.g., gingivitis, mucosal membrane lesions, plaque-induced inflammations, gum bleeds, bone or tooth loss in the oral cavity), or a propensity to develop such conditions.

In general, the measurement of a marker polypeptide in a subject sample is compared with a diagnostic amount present in a reference. A diagnostic amount distinguishes between periodontitis (e.g., gingivitis, mucosal membrane lesions, plaque-induced inflammations, gum bleeds, bone or tooth loss in the oral cavity), or a propensity to develop such conditions and the absence of such condition. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an marker polypeptide or nucleic acid molecule in the subject sample relative to a reference may be used to diagnose periodontitis (e.g., gingivitis, mucosal membrane lesions, plaque-induced inflammations, gum bleeds, bone or tooth loss in the oral cavity), or a propensity to develop such conditions. In one embodiment, the reference is the level of marker polypeptide present in a control sample obtained from a patient that does not have periodontitis (e.g., gingivitis, mucosal membrane lesions, plaque-induced inflammations, gum bleeds, bone or tooth loss in the oral cavity), or a propensity to develop such conditions. In another embodiment, the reference is a baseline level of marker present in a biologic sample derived from a patient prior to, during, or after treatment for periodontitis (e.g., gingivitis, mucosal membrane lesions, plaque-induced inflammations, gum bleeds, bone or tooth loss in the oral cavity), or a propensity to develop such conditions. In yet another embodiment, the reference is a standardized curve.

Accordingly, a marker profile may be obtained from a subject sample and compared to a reference marker profile obtained from a reference population, so that it is possible to classify the subject as belonging to or not belonging to the reference population. The correlation may take into account the presence or absence of the biomarkers in a test sample and the frequency of detection of the same biomarkers in a control. The correlation may take into account both of such factors to facilitate determination of periodontitis (e.g., gingivitis, mucosal membrane lesions, plaque-induced inflammations, gum bleeds, bone or tooth loss in the oral cavity), or a propensity to develop such conditions.

Any marker, individually, is useful in aiding in the determination of the status of periodontitis. First, the selected marker is detected in a subject sample using the methods described herein (e.g. mass spectrometry, immunoassay). Then, the result is compared with a control that distinguishes periodontitis status from non-periodontitis status. As is well understood in the art, the techniques can be adjusted to increase sensitivity or specificity of the diagnostic assay depending on the preference of the diagnostician.

While individual biomarkers are useful diagnostic biomarkers, in some instances, a combination of biomarkers provides greater predictive value than single biomarkers alone. The detection of a plurality of biomarkers (or absence thereof, as the case may be) in a sample can increase the percentage of true positive and true negative diagnoses and decrease the percentage of false positive or false negative diagnoses. Thus, one method provides for the measurement of more than one marker.

Patients identified as having changes in the cobalamin synthesis pathway may be treated using agents that inhibit cobalamin synthesis.

### Cobalamin Synthesis Inhibitors

Cobalamin synthesis inhibitors are useful for the treatment and prevention of periodontal disease, gingivitis, and related disorders. The cobalamin synthesis inhibitor-containing compositions of the invention can be employed to treat periodontitis, alone or in conjunction with other treatments, particularly with an anti-microbial agent, and most commonly with an antibacterial agent.

Cobalamin synthesis inhibitors are known in the art and described herein below (Table 1).

TABLE 1
Genes and Inhibitors of the Cobalamin Synthesis Pathway
ANAEROBIC PATHWAY (Salmonella typhimurium)

| | Gene | | Enzyme Commission | |
|---|---|---|---|---|
| Gene | product | Protein common name | No. | Inhibitor(s) |
| hemB | HemB | Delta-aminolevulinic acid | | |
| | | dehydratase | | |
| hemC | HemC | Porphobilinogen deaminase | | |
| hemD | HemD | Uroporphyrinogen-III synthase | EC_4.2.1.75 | 19-bromo-1- |
| | | | | hydroxymethylbilane, N(D)- |
| | | | | methyl-1-formylbilane |
| cysG | CysG | Siroheme synthase | | |
| cbiK | CbiK | Sirohydrochlorin cobaltochelatase | EC_4.99.1.3 | N-ethylmaleimide, adenylyl- |
| | | | | imidodiphosphate, adenylyl(b,g- |
| | | | | methylene)-diphosphonate, ADP, |
| | | | | PPᵢ, divalent metal ions (Ni²⁺, |
| | | | | Cu²⁺, Zn²⁺, Fe²⁺, Mg²⁺, Mn²⁺) |
| cbiL | CbiL | Precorrin-2 C20-methyltransferase | EC_2.1.1.130 | S-adenosyl-L-homocysteine |
| cbiH | CbiH | Precorrin-3B C17- | EC_2.1.1.131 | S-adenosyl-L-homocysteine |
| | | methyltransferase | | |
| cbiF | CbiF | Precorrin-4 C11-methyltransferase | EC_2.1.1.133 | S-adenosyl-L-homocysteine |
| cbiJ | CbiJ | Precorrin-6X reductase | EC_1.3.1.54 | |
| cbiD | CbiD | Precorrin-6A synthase | EC_2.1.1.152 | |
| cbiG | CbiG | Precorrin-5A hydrolase | EC_3.7.1.12 | |
| cbiE | CbiE | Precorrin-6Y C5,15- | EC_2.1.1.132 | S-adenosyl-L-homocysteine |
| | | methyltransferase | | |
| cbiT | CbiT | Precorrin-6Y C5,15- | EC_2.1.1.132 | S-adenosyl-L-homocysteine |
| | | methyltransferase | | |
| cbiC | CbiC | Precorrin-8X methylmutase | EC_5.4.1.2 | |
| cbiA | CbiA | Cobyrinic acid a,c-diamide | EC_6.3.5.11 | |
| | | synthase | | |
| cobA | CobA | Cob(I)yrinic acid a,c-diamide | EC_2.5.1.17 | tripolyphosphate/sodium |
| | | adenosyltransferase | | triphosphate (GRAS status), |
| | | | | hydrogenobyrinic acid a,c- |
| | | | | diamide |
| cbiP | CbiP | Cobyric acid synthase | EC_6.3.5.10 | |
| cbiB | CbiB | Adenosylcobinamide-phosphate | EC_6.3.1.10 | |
| | | synthase | | |
| cobD | CobD | Threonine-phosphate | EC_4.1.1.81 | |
| | | decarboxylase | | |
| cobU | CobU | Adenosylcobinamide | EC_2.7.7.62 | |
| | | kinase/Adenosylcobinamide- | | |
| | | phosphate guanylyltransferase | | |
| cobS | CobS | Cobalamin-5-phosphate synthase | EC_2.7.8.26 | |
| cobT | CobT | Nicotinate-nucleotide-- | EC_2.4.2.21 | |
| | | dimethylbenzimidazole | | |
| | | phosphoribosyltransferase | | |

Without being bound to theory, a cobalamin synthesis inhibitor specifically binds and/or reduces the level, expression, and/or activity of a cobalamin synthesis nucleic acid or gene product. Cobalamin synthesis synthesis genes and gene products are known in the art and are described herein below (Table 2).

TABLE 2
Genes and Gene Products of the Cobalamin Synthesis Pathway
AEROBIC PATHWAY

| | Gene | |
|---|---|---|
| Gene | product | Protein common name |
| hemB | HemB | Delta-aminolevulinic acid dehydratase |
| hemC | HemC | Porphobilinogen deaminase |
| hemD | HemD | Uroporphyrinogen-III synthase |
| cobA | CobA | Siroheme synthase |
| cobI | CobI | Precorrin-2 C20-methyltransferase |
| cobG | CobG | Precorrin-3B synthase |
| cobJ | CobJ | Precorrin-3B C17-methyltransferase |
| cobM | CobM | Precorrin-4 C11-methyltransferase |
| cobF | CobF | Precorrin-6A synthase |
| cobK | CobK | Precorrin-6X reductase |
| cobL | CobL | Precorrin-6Y C5,15-methyltransferase |
| cobH | CobH | Precorrin-8X methylmutase |
| cobB | CobB | Cobyrinic acid a,c-diamide synthase |
| cobN | CobN | Cobaltochelatase |
| cobS | CobS | Adenosylcobinamide-GDP ribazoletransferase |
| cobT | CobT | Nicotinate-nucleotide--dimethylbenzimidazole |
| | | phosphoribosyltransferase |
| cobO | CobO | Cob(I)yrinic acid a,c-diamide adenosyltransferase |
| cobQ | CobQ | Cobyric acid synthase |
| cobD | CobD | Threonine-phosphate decarboxylase |
| cobC | CobC | Threonine-phosphate decarboxylase |
| protein a | Protein a | — |
| cobP | CobP | Adenosylcobinamide kinase/ |
| | | Adenosylcobinamide- |
| | | phosphate guanylyltransferase |
| cobV | CobV | Cobalamin-5-phosphate synthase |
| cobU | CobU | Adenosylcobinamide kinase/ |
| | | Adenosylcobinamide- |
| | | phosphate guanylyltransferase |

### Structures of Cobalamin Synthesis Inhibitors

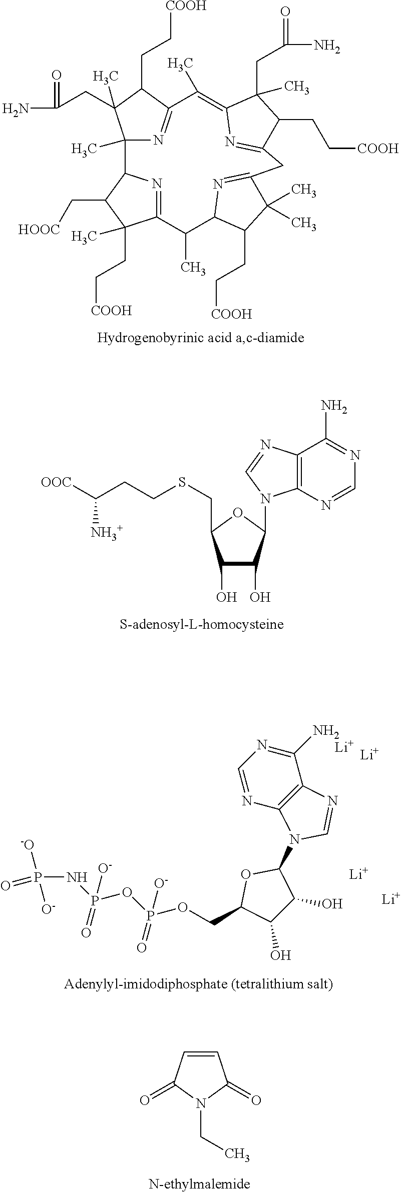

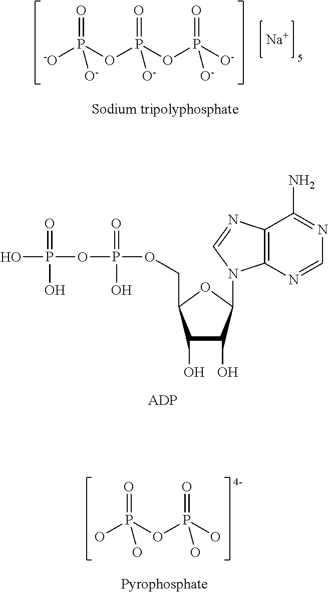

### Methods of Delivery

Cobalamin synthesis inhibitors may be delivered using any oral formulation known in the art as described herein below.

Oral Sprays, Rinses, and Emulsions

Cobalamin system inhibitors (Tables 1 and 2) are desirably administered to an oral cavity (e.g., teeth, gums, mucosal membranes, tongue, periodontal pockets) using compositions of the invention. Spray systems are particularly useful for delivering therapeutics to the oral cavity. Suitable spray delivery systems include both pressurized and non-pressurized (pump actuated) delivery devices. The cobalamin synthesis inhibitor-containing solution, delivered as an oral spray, is preferably an aqueous solution; however, organic and inorganic components, emulsifiers, excipients, and agents that enhance the organoleptic properties (i.e., flavoring agents or odorants) may be included. Optionally, the solution may contain a preservative that prevents microbial growth (i.e., methyl paraben). Although water itself may make up the entire carrier, typical liquid spray formulations contain a co-solvent, for example, propylene glycol, corn syrup, glycerin, sorbitol solution and the like, to assist solubilization and incorporation of water-insoluble ingredients. In general, therefore, the compositions of this invention preferably contain from about 1-95% v/v and, most preferably, about 5-50% v/v, of the co-solvent. When prepared as an spray, patients typically self-administer 1-5 times per day. The spray delivery system is normally designed to deliver 50-100 .mu.1 per actuation, and therapy may require 1-5 actuations per dose. The rheological properties of the spray formulation are optimized to allow shear and atomization for droplet formation. Additionally, the spray delivery device is designed to create a droplet size which promotes retention on mucosal surfaces of the oral cavity and minimize respiratory exposure.

Compositions suitable for oral sprays can also be formulated as an oral rinse or mouthwash. Administration of cobalamin synthesis inhibitors using these formulations is typically done by swishing, gargling, or rinsing the oral cavity with the formulation.

### Ointments, Pastes, and Gels

Lesions of the oral cavity caused by periodontal disease or trauma are amenable to cobalamin synthesis inhibitor therapy delivered as an ointment, paste, or gel. The viscous nature of these types of preparations allows for direct application into the wound site. Optionally, the wound site can be covered with a dressing to retain the cobalamin synthesis inhibitor-containing composition, protect the lesion from trauma, and/or absorb exudate. As discussed further below, these preparations are particularly useful to restore integrity of the mucous membrane and gum of the oral cavity following traumatic surgical procedures such as, for example, tooth extraction, tissue biopsy, or a tumor resection. Such viscous formulations may also have a local barrier effect thereby reducing irritation and pain.

### Mucoadhesives

A mucoadhesive excipient can be added to any of the previously described pharmaceutical compositions. The mucoadhesive formulations coat the oral cavity providing protection, inhibiting irritation, and accelerating healing of inflamed or damaged tissue. Mucoadhesive formulations also promote prolonged contact of the cobalamin synthesis inhibitor with the mucosal epithelium. Mucoadhesive formulations suitable for use in pharmaceutical preparations delivered by mouth are well known in the art (e.g., U.S. Pat. No. 5,458,879). Particularly useful mucoadhesives are hydrogels composed of about 0.05-20% of a water-soluble polymer such as, for example, poly(ethylene oxide), poly(ethylene glycol), poly(vinyl alcohol), poly(vinyl pyrrolidine), poly(acrylic acid), poly(hydroxy ethyl methacrylate), hydroxyethyl ethyl cellulose, hydroxy ethyl cellulose, chitosan, and mixtures thereof. These polymeric formulations can also contain a dispersant such as sodium carboxymethyl cellulose (0.5-5.0%).

Other preferred mucoadhesive excipients for liquid compositions are ones that allow the composition to be administered as a flowable liquid but will cause the composition to gel in the oral cavity, thereby providing a bioadhesive effect which acts to hold the therapeutic agents at the lesion site for an extended period of time. The anionic polysaccharides pectin and gellan are examples of materials which when formulated into a suitable composition will gel in the oral cavity, owing to the presence of cations in the mucosal and salivary fluids. The liquid compositions containing pectin or gellan will typically consist of 0.01-20% w/v of the pectin or gellan in water or an aqueous buffer system.

Other useful compositions which promote mucoadhesion and prolonged therapeutic retention in the oral cavity are colloidal dispersions containing 2-50% colloidal particles such as silica or titanium dioxide. Such formulations form as a flowable liquid with low viscosity suitable as a mouthwash or for generating a fine mist. However, the particles interact with glycoprotein, especially mucin, transforming the liquid into a viscous gel, providing effective mucoadhesion (e.g., U.S. Pat. Nos. 5,993,846 and 6,319,513).

### Bioerodable Film Delivery Devices

The most simple bioerodable devices contain the therapeutic agent(s) incorporated into a solid, usually lipid-containing, film or tablet. The device is formulated to remain solid at room temperature, but melt at body temperature, releasing the incorporated therapeutics. Suitable formulations of this type include, for example, cocoa butter.

Polymeric film devices provide several advantages for therapeutic delivery to the oral cavity. Unlike rinses, pastes, gels, and other flowable compositions, a film device can reside for prolonged periods of time (i.e., hours to days) in the oral cavity and provide sustained release throughout its residency. Typically, the film is partially or completely bioerodable and contains a mucoadhesive layer to fasten the film to the oral mucosa. Film devices, in addition to its use for delivering therapeutics, can also provide protection against mechanical injury or microbial infection of a lesion site. This physical barrier function is particularly advantageous when treating conditions such as periodontal disease. Additionally, as discussed further below, a film device can be used to release cobalamin synthesis inhibitor therapy directly onto the underlying mucosa, into the lumen of the oral cavity, or a combination of both.

Film devices consist of at least two layers; a mucoadhesive layer suitable for attaching the film to the oral mucosa and a bulk layer which contains the active therapeutic(s). Many suitable mucoadhesives are known in the art and are discussed above. Optionally, one or more therapeutics can also be provided in the adhesive layer.

The bulk layer of the composite delivery device may be made of one or more bioerodable polymeric materials. Suitable polymers include, for example, starch, gelatin, polyethylene glycol, polypropylene glycol, polyethylene oxide, copolymers of ethylene oxide and propylene oxide, copolymers of polyethylene glycol and polypropylene glycol, polytetramethylene glycol, polyether urethane, hydroxyethyl cellulose, ethyl cellulose, hydroxypropyl cellulose, hydroxypropylmethyl cellulose, alginate, collagen, polylactide, poly(lactide-co-glycolide) (PLGA), calcium polycarbophil, polyethymethacrylate, cellulose acetate, propylene glycol, polyacrylic acid, crosslinked polyacrylic acid, hydroxyethyl methacrylate/methyl methacrylate copolymer, silicon/ethyl cellulose/polyethylene glycol, urethane polyacrylate, polystyrene, polysulfone, polycarbonate, polyorthoesters, polyanhydrides, poly(amino acids), partially and completely hydrolyzed alkylene-vinyl acetate copolymers, polyvinyl chloride, polymers of polyvinyl acetate, polyvinyl alkyl ethers, styrene acrylonitrile copolymers, poly(ethylene terphthalate), polyalkylenes, poly(vinyl imidazole), polyesters and combinations of two or more of these polymers.

A particularly useful bulk layer polymer consists of PLGA and ethyl cellulose. PLGA is bioerodable and can be formulated to degrade over a wide range of conditions and rates. Ethyl cellulose is a water-insoluble polymer that can act as a plasticizer for the PLGA when a film is formed, but will be eroded in a bodily fluid. Due to its water-insolubility, it also has an effect on the degree and rate of swelling of the resultant film.

An optional third layer which is impermeable to the cobalamin synthesis inhibitor can also be added to the wafer. Preferably, this barrier layer is also bioerodable. Suitable barrier layer polymers include ethyl cellulose, poly(acrylic acid), or other polyelectrolytes. In one configuration, the barrier layer is placed on the opposite side of the bulk layer relative to the adhesive layer, thereby directing the released therapeutic agent onto the contacted epithelium rather than being diluted in the lumenal fluid of the oral cavity. This configuration is particularly useful for treating discrete lesions of the tongue, sublingual tissue, or buccal mucosa. In an alternative configuration of the film device, the barrier layer is placed between the bulk layer and the adhesive layer. This configuration directs therapeutic release into the lumen of the oral cavity and is useful for treating more diffuse lesions of the tongue and oral cavity. The configuration is also useful for delivering therapeutics which are cytotoxic when administered at high concentrations because it has the effect of shielding the underlying tissue from direct contact with the therapeutic-containing film.

### Chewable Tablets, Lozenges, and Confectionaries

Preparing a cobalamin synthesis inhibitor-containing composition as a chewable tablet, lozenge, or a confectionary such as chewing gum provides several advantages to traditional drug delivery vehicles. First, prolonged contact and sustained release at the target site (oral cavity) is achieved. Second, such formulations often results in higher patient compliance, especially when administering cobalamin synthesis inhibitors to children.

Formulations for chewable tablets are well known and typically contain a base of sugar, starch, or lipid and a flavoring agent.

The incorporation of therapeutics into chewing gum and other confectionary style formulations is known in the art (e.g., U.S. Pat. No. 5,858,391).

### Combination Therapies

Cobalamin synthesis inhibitors may be used in combination anti-bacterial agents. Examples of antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin; gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin.

### Analgesics and Anesthetics

Periodontitis is often accompanied by painful lesions of the oral mucosal membrane and bleeding gums. Any of the commonly used topical analgesics can be used in combination with the cobalamin synthesis inhibitors. Examples of other useful anesthetics include procaine, lidocaine, tetracaine, dibucaine, benzocaine, p-buthylaminobenzoic acid 2-(diethylamino) ethyl ester HCl, mepivacaine, piperocaine, and dyclonine.

Other analgesics include opioids such as, for example, morphine, codeine, hydrocodone, and oxycodone. Any of these analgesics may also be co-formulated with other compounds having analgesic or anti-inflammatory properties, such as acetaminophen, aspirin, and ibuprofen.

### Kits

In one aspect, the invention provides kits for evaluating, such as monitoring the development of or diagnosing, periodontitis (e.g., gingivitis, mucosal membrane lesions, plaque-induced inflammations, gum bleeds, bone or tooth loss in the oral cavity), or a propensity to develop such conditions, wherein the kits can be used to detect genes of the cobalamin synthesis pathway or potassium transporters described herein. For example, the kits can be used to detect any one or more of the biomarkers potentially differentially present in samples of test subjects vs. normal subjects (e.g., proteins critical for cobalamin synthesis or potassium ion transport) or control proteins. If desired a kit includes any one or more of the following: capture molecules that bind proteins involved in cobalamin synthesis or potassium ion transport. The kits have many applications. For example, the kits can be used to differentiate if a subject has periodontal disease, has a propensity to develop periodontal disease or has a negative diagnosis. In another embodiment, kits are provided for aiding the diagnosis of periodontal disease or the diagnosis of a specific type of plaque-induced inflammation or related condition such as, for example, gingivitis, and other mucosal membrane lesions, gum bleeds, or tooth and bone-loss in the oral cavity. The kits can also be used to identify agents that modulate expression of one or more of the herein-described biomarkers in in vitro or in vivo animal models for periodontal disease.

The kits may include instructions for the assay, reagents, testing equipment (test tubes, reaction vessels, needles, syringes, etc.), standards for calibrating the assay, and/or equipment provided or used to conduct the assay. The instructions provided in a kit according to the invention may be directed to suitable operational parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of periodontal disease or the diagnosis of a specific type of plaque-induced inflammation or related condition such as, for example, gingivitis, and other mucosal membrane lesions, gum bleeds or bone or tooth loss in the oral cavity.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Phylogenetic Differences Between Sites in Metagenome and Metatranscriptome Composition

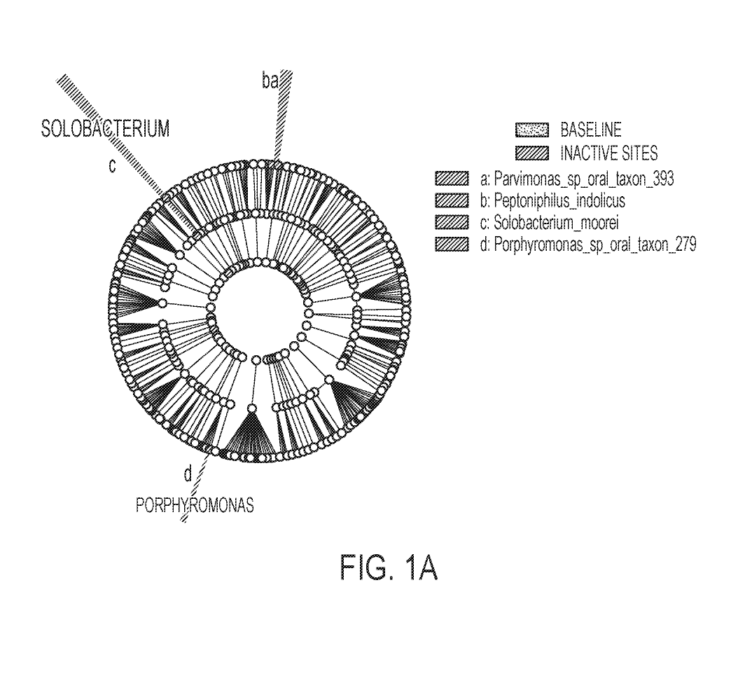

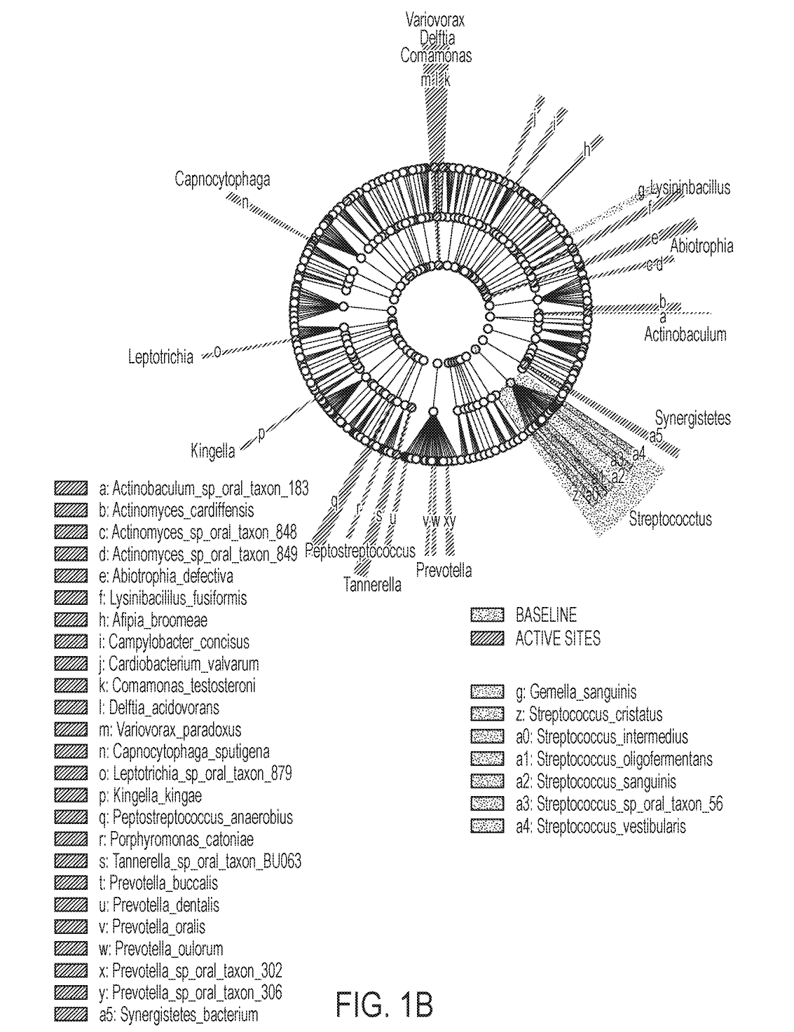

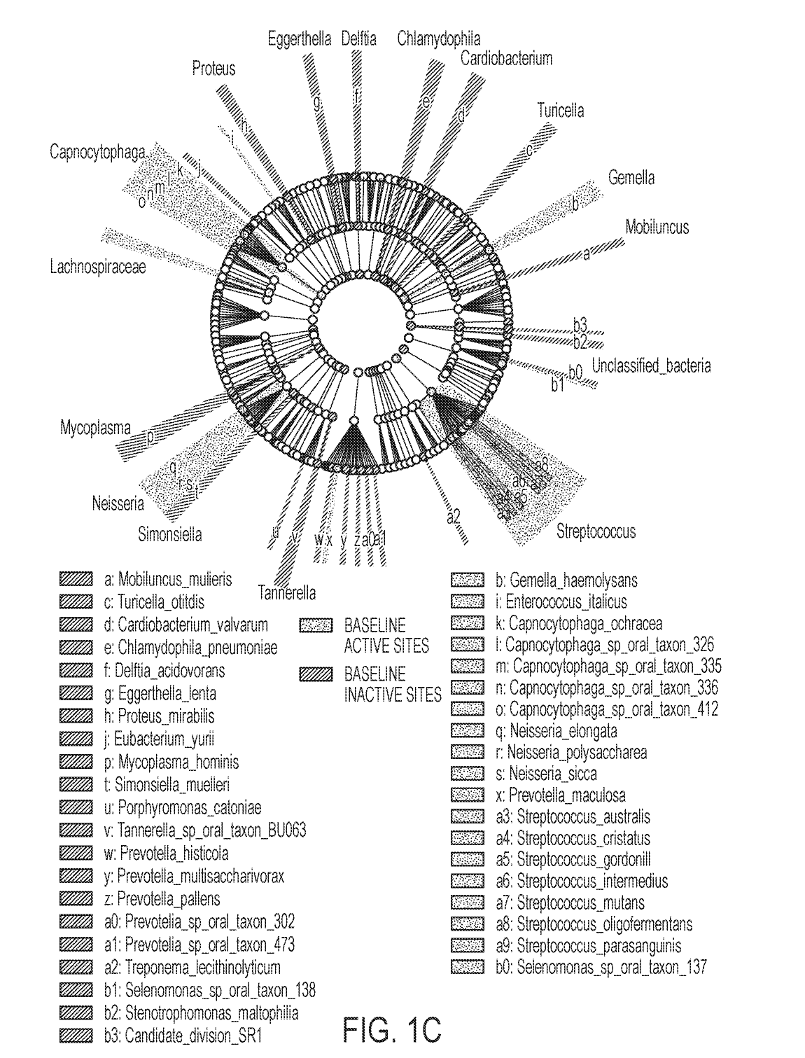

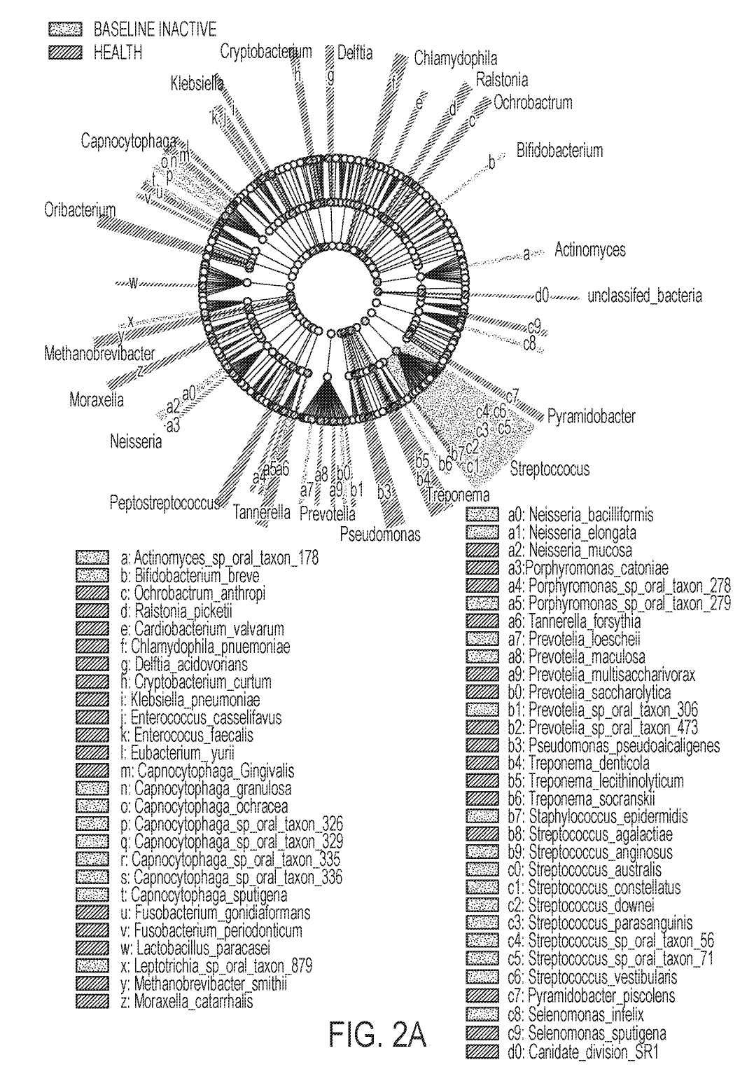

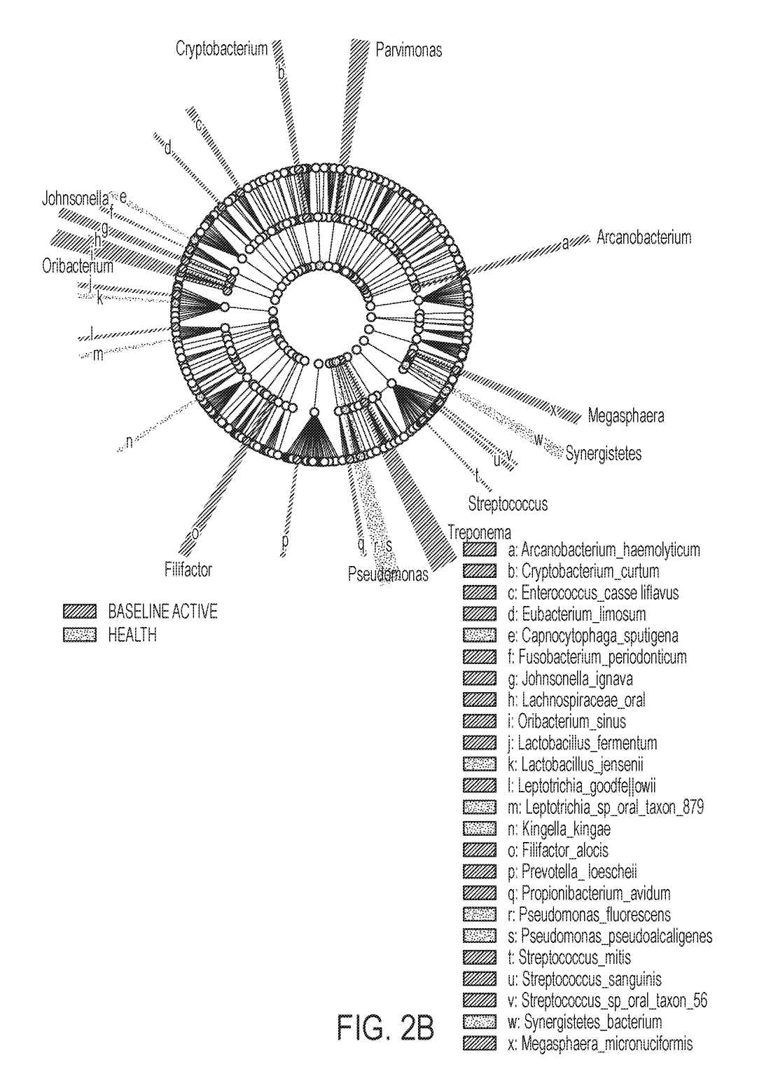

The comparison of phylogenetic assignments of the metagenome are presented in FIGS. 1A-1C. Two major observations could be derived from these results. First, changes in the metagenome of periodontitis non-progressing sites were minor, only 4 species were significantly more abundant in the community at the end point of the presented study (FIG. 1A). Second, differences in periodontitis progressing sites were more significant (FIG. 1B) and in those Streptococci dominated the community at baseline compared with the periodontitis progressing community. What was more striking was the complete rearrangement at the metagenome level between the baselines of sites that did not progress versus sites that did progress (FIG. 1C). Additionally, the metagenome of baseline from periodontital disease progressing sites and non-progressing sites was compared with samples from healthy sites of periodontally healthy individuals from a previous study (Duran-Pinedo et al., ISME J, 8:1659-1672, 2014). The metagenome composition of both baseline communities is altered when compared to healthy communities (FIGS. 2A and 2B). Streptococcus species (spp.) were more abundant in health than in either of the 2 baselines, while known periodontal pathogens such as Treponema denticola and Tannerella forsythia were more abundant in the baseline samples (FIG. 2A). However, Streptococcus spp. were more abundant in the baseline from periodontitis progressing sites than in healthy samples (FIG. 2B).

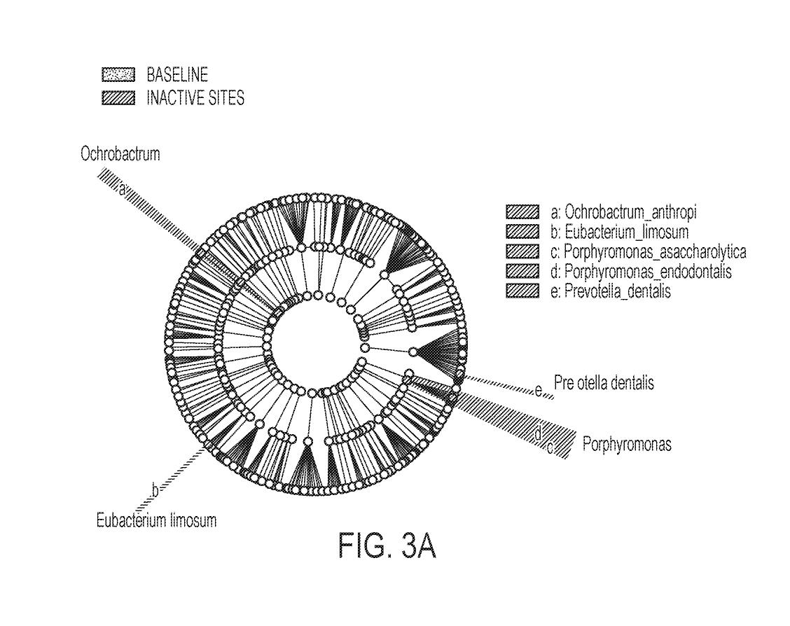

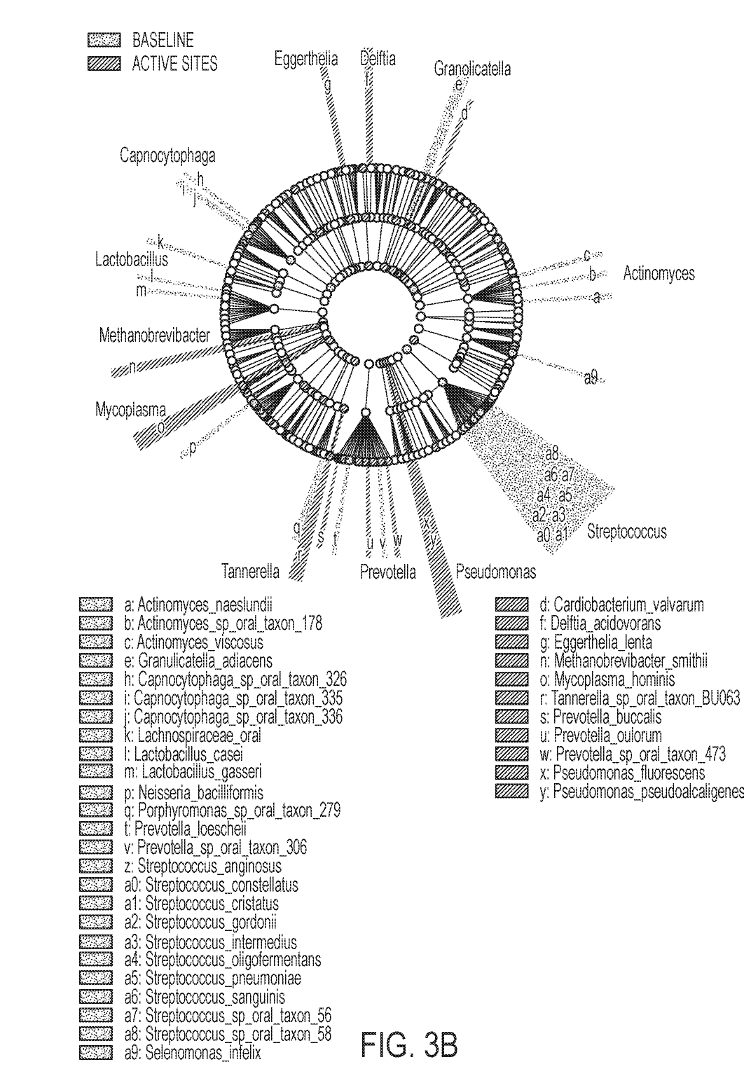

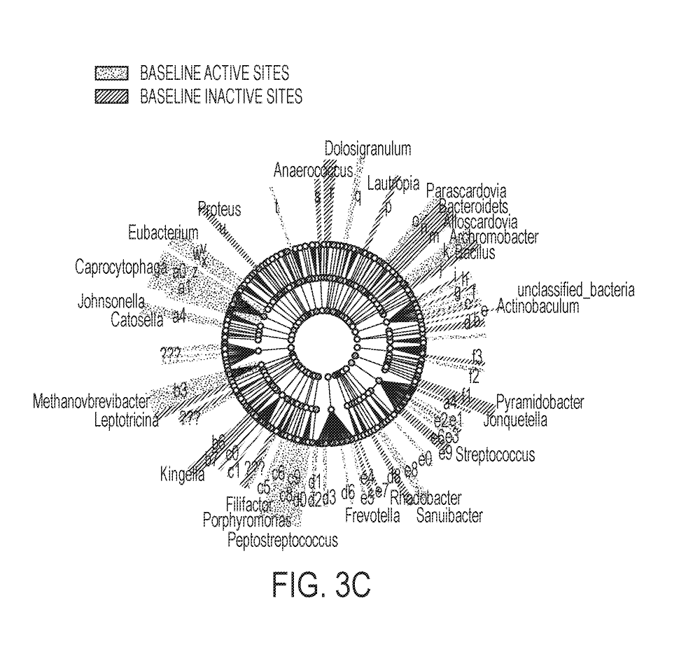

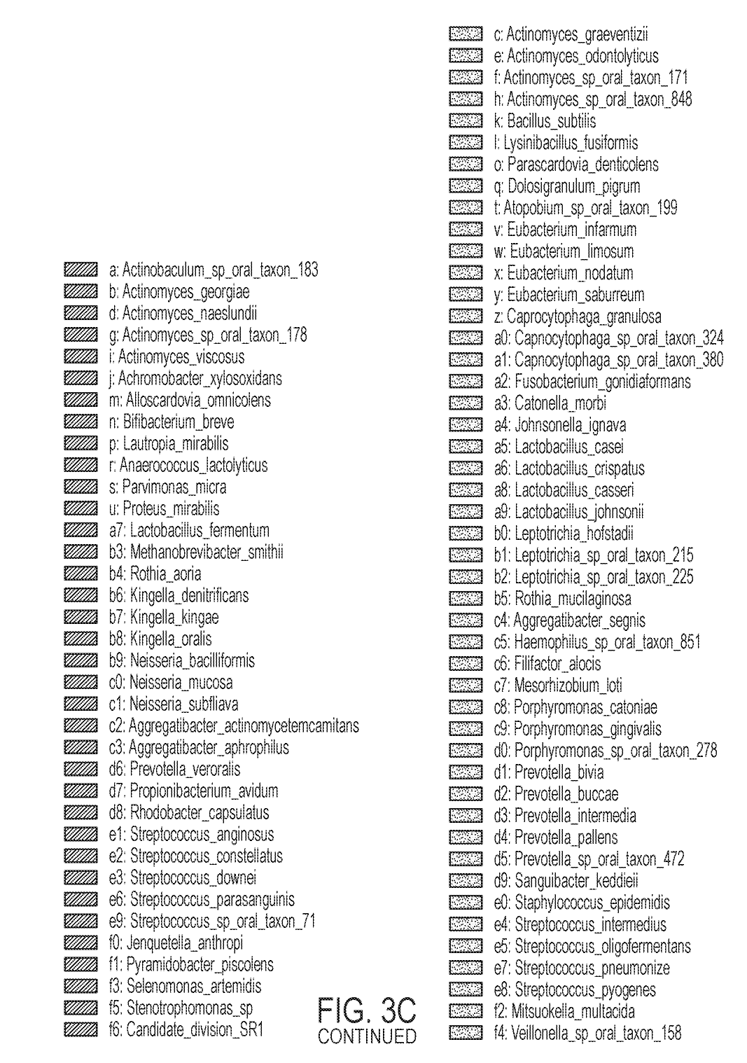

Next the fraction of the active community under the different conditions studied was examined. To perform these analysis the metatranscriptome results were first normalized by the relative abundance of the different species to obtain differences in expression due to real changes in levels of gene expression and not to an increase in numbers of certain members of the community. Species frequencies were estimated using Genome Abundance Similarity Correction (GASiC, Lindner et al., Nucleic Acids Res.; 41:e10, 2013). FIGS. 3A-3C show the results of these analyses. As in the case of the metagenome only a few species were significantly more active in the non-progressing sites at the end of the study (FIG. 3A). When periodontitis progressing sites to their baseline, and baselines from periodontitis progressing and non-progressing sites were compared, the differences were larger (FIGS. 3B and 3C). Streptococcus species (spp.) dominated the activity of the community at baseline of periodontitis progressing sites (FIG. 3B). Moreover, some members of the orange P. gingivalis, several members of the orange complex including P. intermedia and E. nodatum, and the putative periodontopathogen Filifactor alocis were more active at the baseline of active sites than the baseline of non-progressing sites (FIG. 3C).

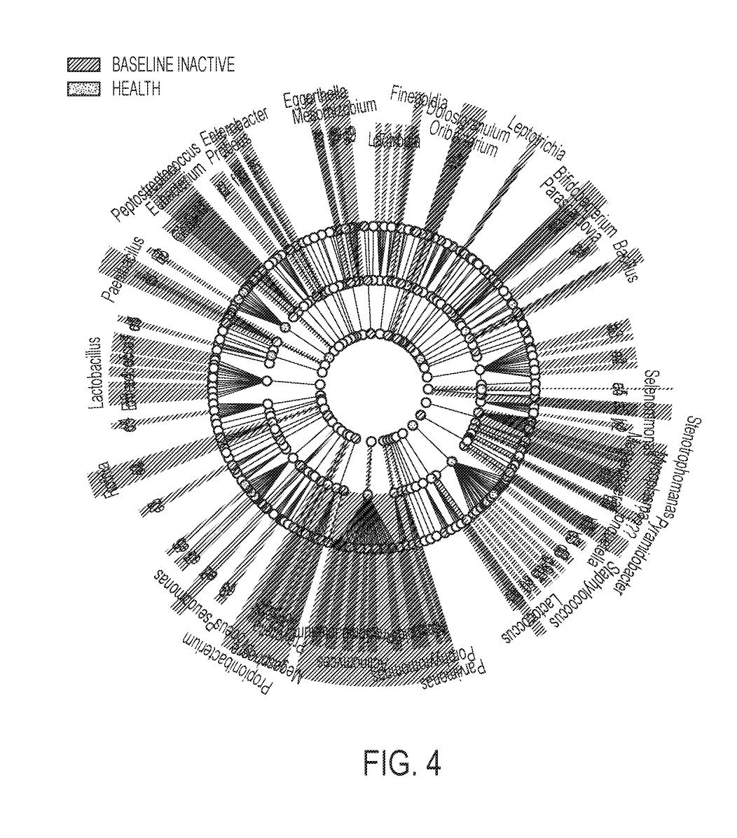

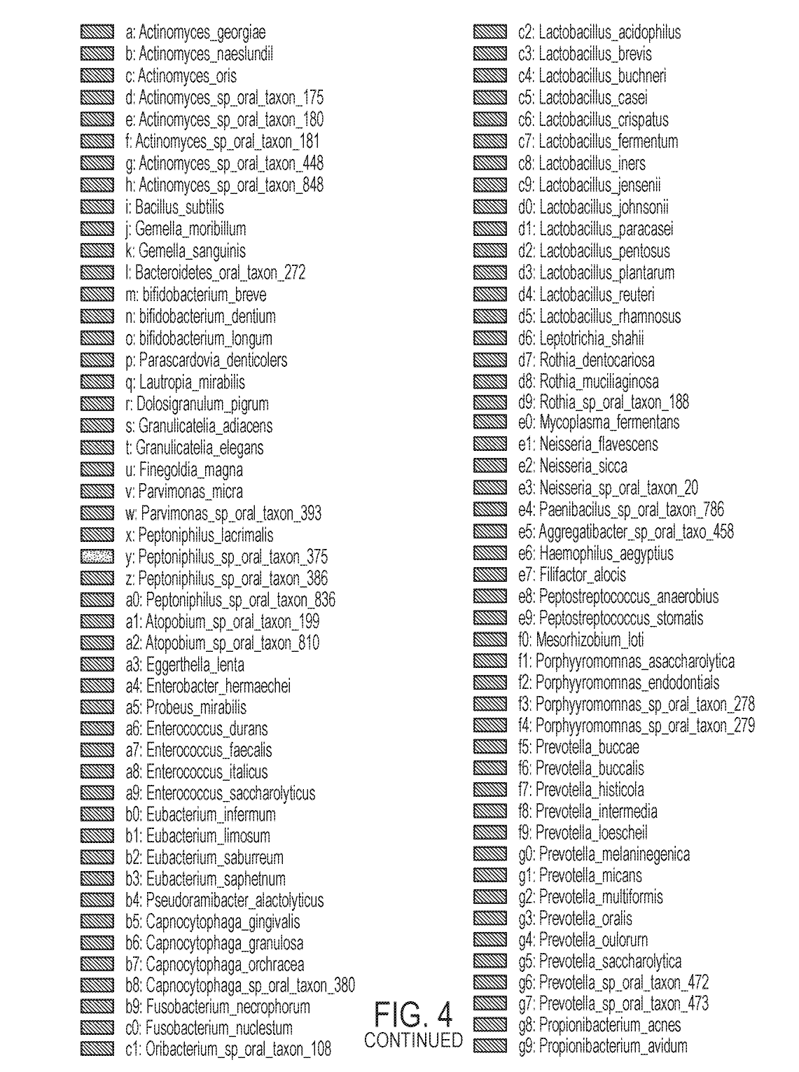

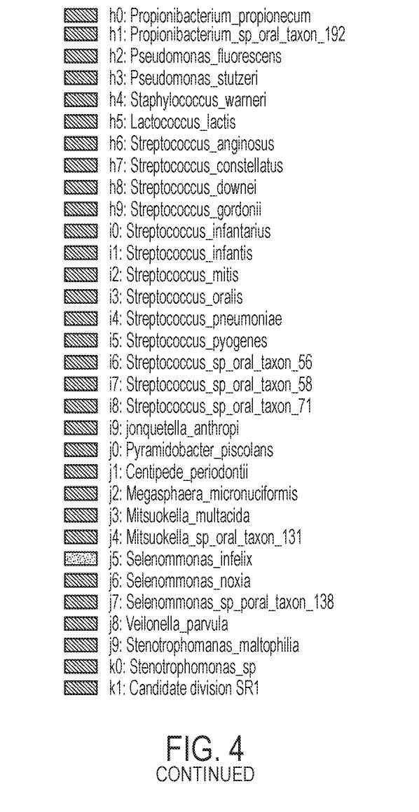

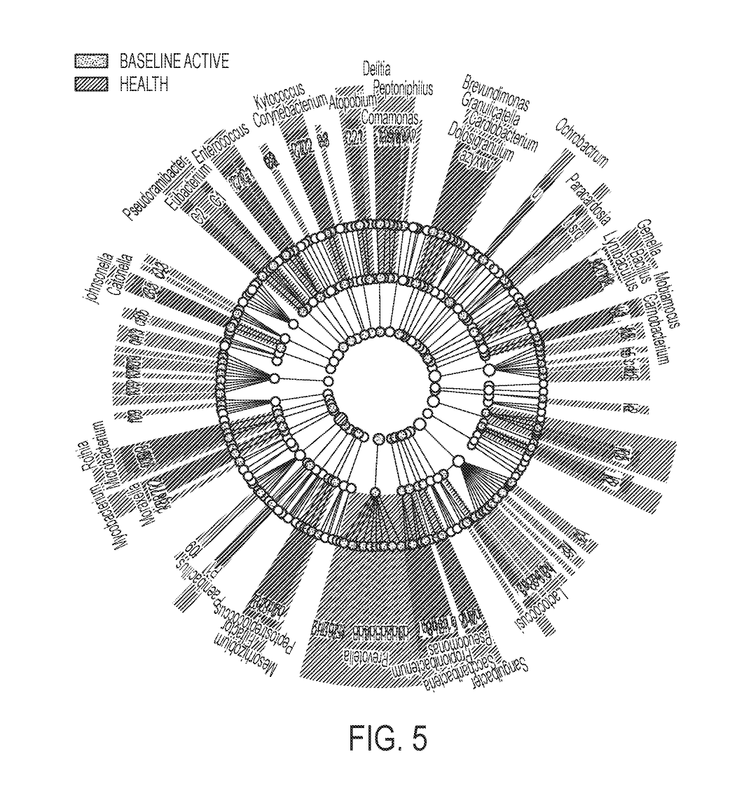

As with the metagenome, the results of activity at baseline and time of periodontital disease progression were also compared with the activity of the community in healthy samples. When baseline of non-progressing sites to healthy sites was compared the first obvious result was that a larger fraction of the community was more active in the baseline samples than in health (FIG. 4). The same could be said for the baseline samples of sites that progressed; a large fraction of the community was significantly more active than the subgingival communities in health (FIG. 5).

### Example 2

### Community-Wide Changes in Patterns of Gene Expression in Non-Progressing and Progressing Sites During Periodontitis Progression

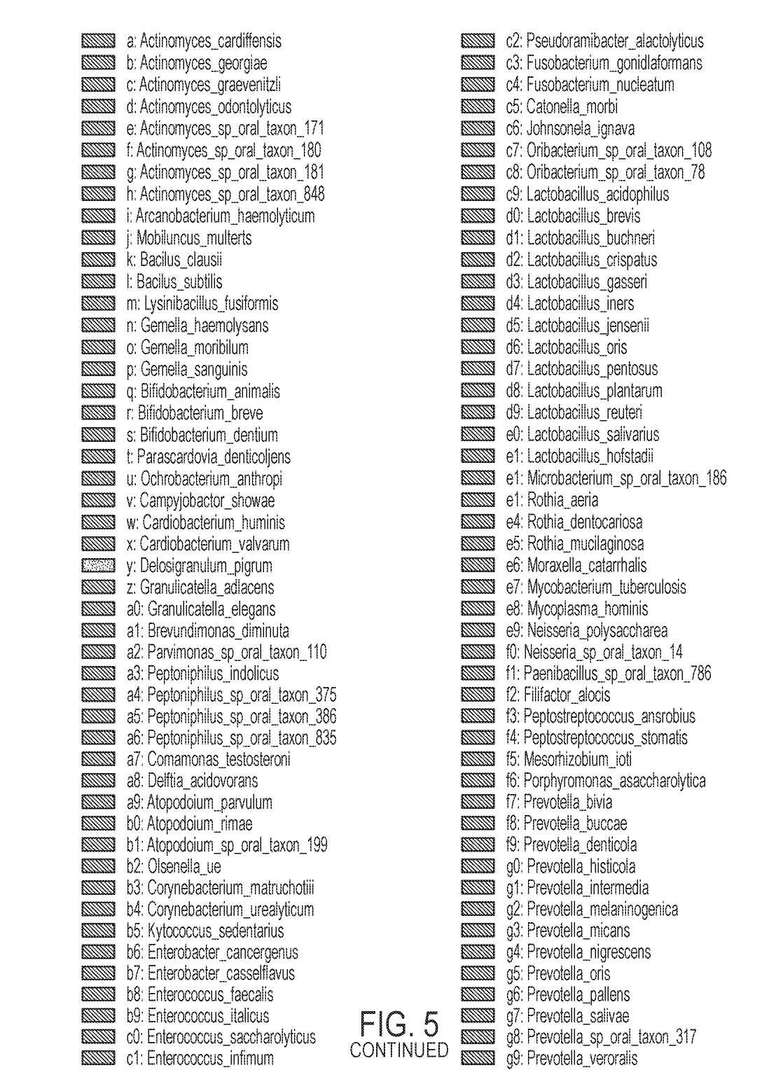

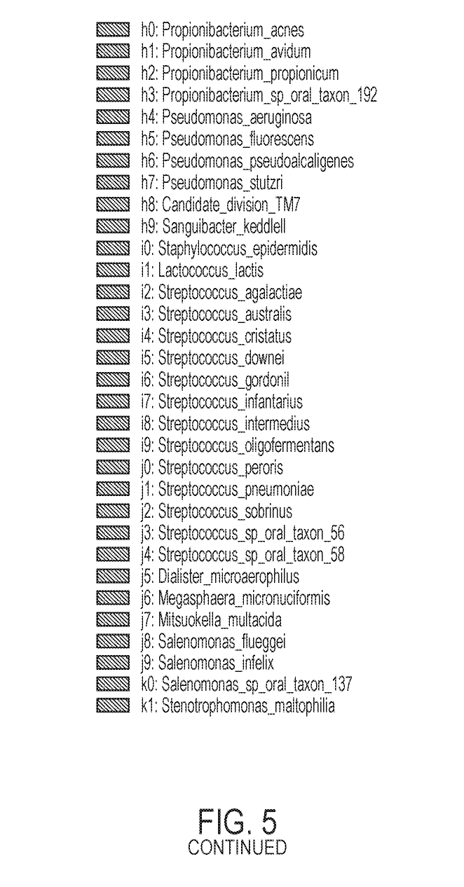

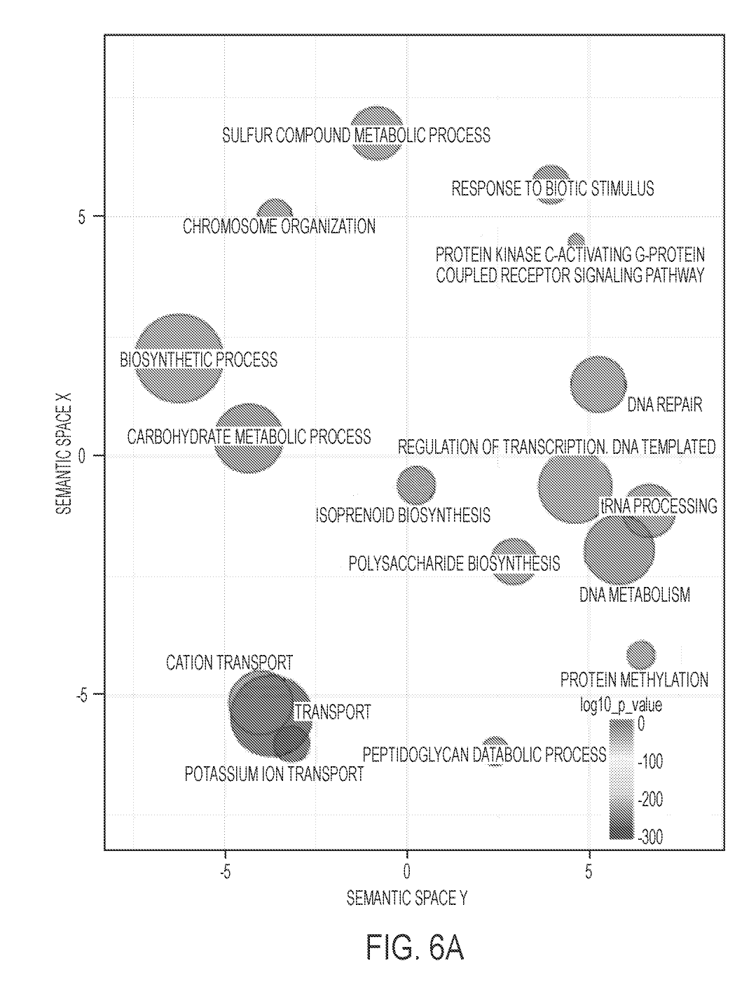

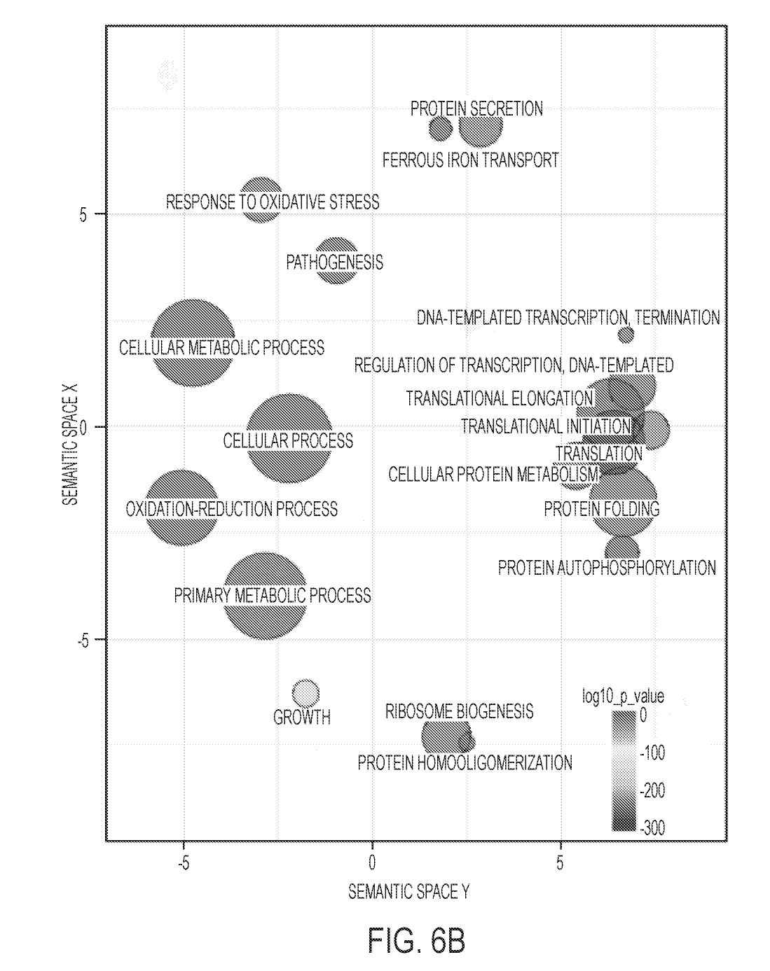

First differences in gene expression between baseline and periodontitis progression were characterized on sites that showed progressing patterns throughout the whole period of the present study (1 year). The global behavior of the community was analyzed by identifying enrichment of Gene Ontology (GO) terms. These results represent global changes in the community and could be due to over-expression of certain genes as well as increase in members of the community whose contribution to the observed activities was now higher due to their larger number. Potassium and amino-acid transport, peptidoglycan catabolism, isoprenoid biosynthesis, polysaccharide biosynthesis and protein kinase C-activating G-protein coupled receptor signaling pathway were over-represented activities at baseline when compared to the end point of progression (FIG. 6A). Over-represented activities at sampling time in periodontitis progressing sites are shown in FIG. 6B. An over-representation of pathogenesis associated GO terms as well as activities related to response to oxidative stress were observed.

Hit counts were normalized against species frequencies estimated using Genome Abundance Similarity Correction (GASiC) (Lindner et al., Nucleic Acids Res.; 41:e10, 2013). These normalized counts represented actual over-expression at the species level and not just increase in the total number of individuals in the community. Interestingly, the list of differentially expressed (DE) genes before and after normalization was very similar (FIG. 7). 132,351 of the DE genes were identical in both, normalized and no-normalized gene sets. Only 412 DE genes were identified in the normalized list and only 6,126 in the original set of DE genes. These results indicate that most of the differences observed were due to changes in gene expression at the species level rather than changes in the numbers of the members of the community.

When the expression profiles of baseline and follow-up samples from non progressing sites (i.e. sites that did not change based on clinical attachment levels, CAL) were compared no gene was identified as differentially expressed, indicating that clinically stable sites did not have significant changes in gene expression during the period those sites were studied.

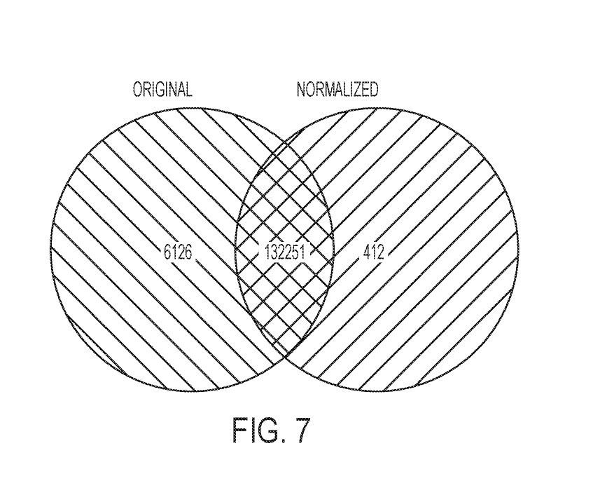

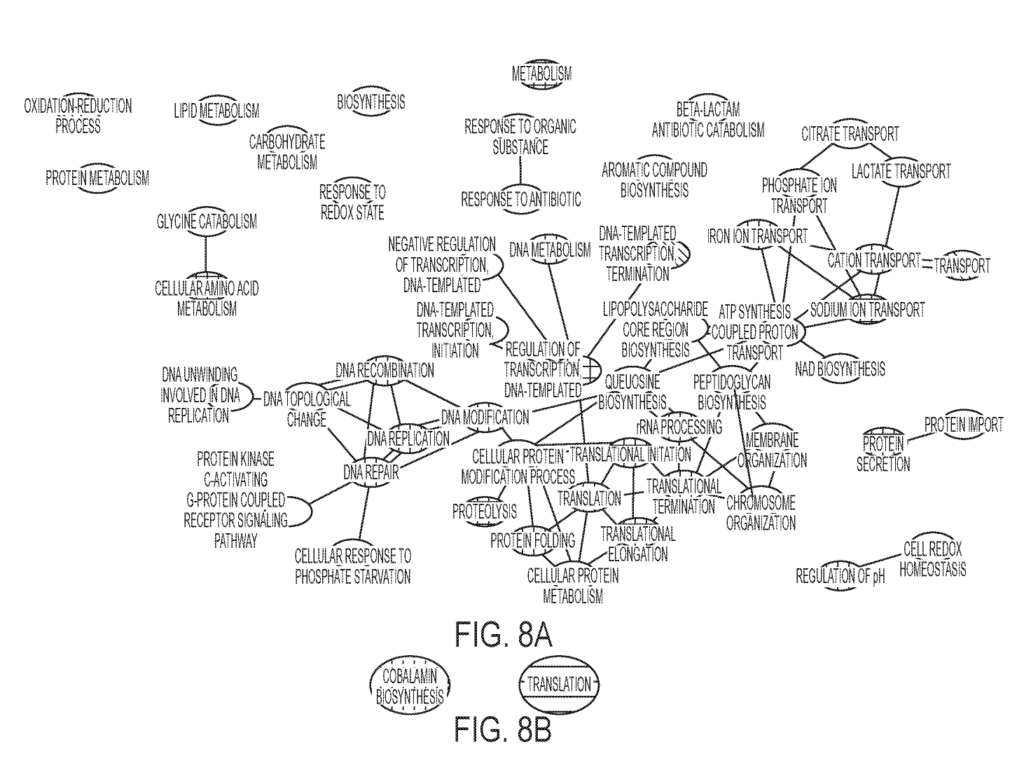

Changes in gene expression profiles in major periodontal pathogens members of the red complex (P. gingivalis, T. denticola and T. forsythia) during periodontal disease progression showed that up-regulated genes belonged to GO terms associated with transport (iron ion, cation, and phosphate), proteolysis, protein kinase C-activating G-protein coupled receptor signaling pathway and response to antibiotic (FIG. 8A), while down-regulated genes belonged to GO terms associated with cobalamin (vitamin B12) biosynthesis (FIG. 8B). Individually it was observed that Treponema denticola up-regulated genes related to flagella biosynthesis (flaA, flaG, fliQ and fliW), oligopeptide ATP-binding cassette (ABC) transporters, and a large number of hypothetical proteins (Table 5). Tannerella forsythia and Porphyromonas gingivalis both up-regulated different TonB-dependent receptors, genes involved in iron transport (ferric uptake siderophores and ferrous iron transport protein B), a large number of peptidases and proteases including ClpB, genes associated with aerotolerance (Bacteroides aerotolerance operon batA-E and moxR-like ATPase of the aerotolerance operon), and Clustered regularly interspaced short palindromic repeats (CRISPR)- associated genes and cobalt-zinc-cadmium resistance proteins (Table 5).

Finally, P. gingivalis specifically up-regulated large number of genes related to biotin synthesis (bioC and bioG), capsular polysaccharide biosynthesis proteins and large number of proteins of conjugative transposons (traA, traB, traE, traF, traG, tral, traf, traK, traL, traM, traN, traO, traP and traQ) and transposases (ISPg2, ISPg3, ISPg4, ISPg5 176 and ISPg6). T. forsythia specifically up-regulated transposases (IS116, IS110, IS902 and IS4 families) and large numbers of different homologs of SusC and SusD family proteins, involved in polysaccharide binding. Regarding down-regulated proteins of the red complex most of them were hypothetical in all three of its members (Table 5).

Profiles of expression of the members of the orange complex were very similar to the ones from the red complex. They up-regulated different TonB-dependent receptors, a large number of peptidases and proteases including ClpB, genes associated with aerotolerance (Bacteroides aerotolerance operon batA-E i and moxR-like ATPase of the aerotolerance operon in P. intermedia and P. nigrescens), genes involved in iron transport (ferric uptake siderophores and ferrous iron transport protein B), hemolysins, cluster regularly interspaced short palindromic repeats (CRISPR)—associated genes (in C. gracilis, C. rectus, C. showae, P. nigrescens and S. constellatus) and chaperones GroEL and GroES and GrpE (Table 5). As in the case of P. gingivalis, both Prevotella, P. intermedia and P. nigrescens up-regulated a large number of genes from conjugative transposons (traA, traB, traD, traE, traF, traG, tral, traJ, traK, traL traM, traN, traO and traQ) (Table 5).).

### Example 3

### Baseline Comparison of Metatranscriptomic Profiles from Active Vs. Non-Progressing Sites

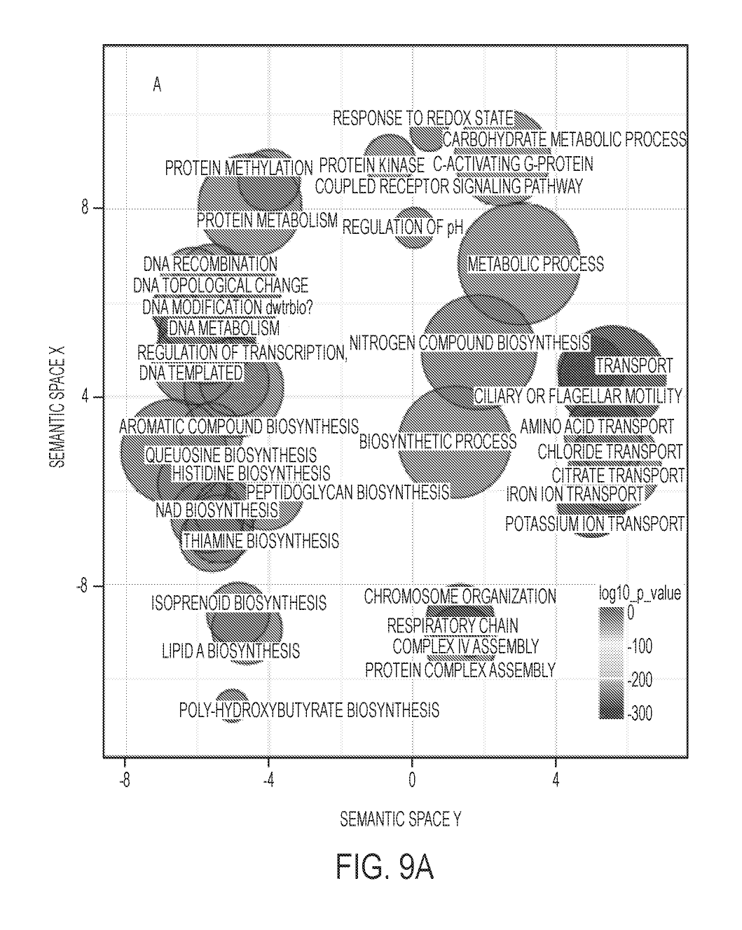

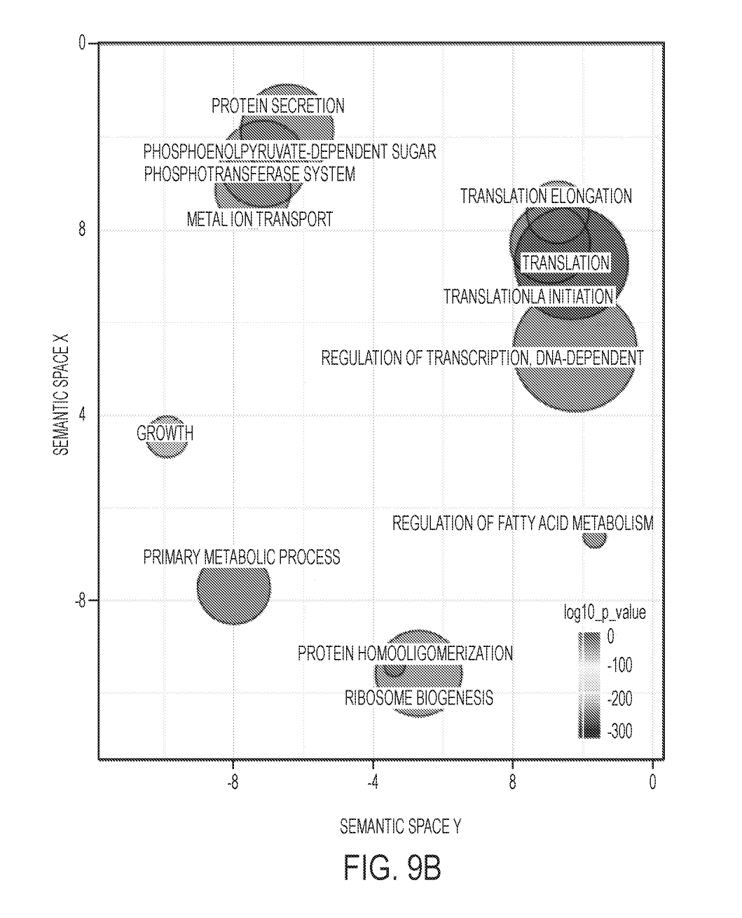

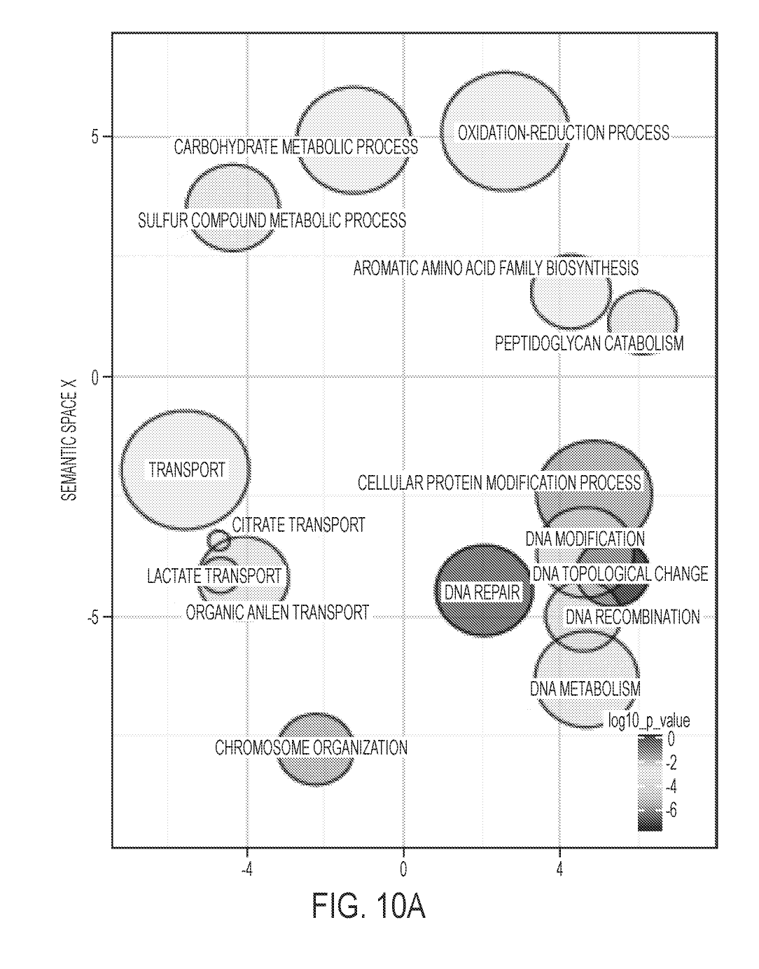

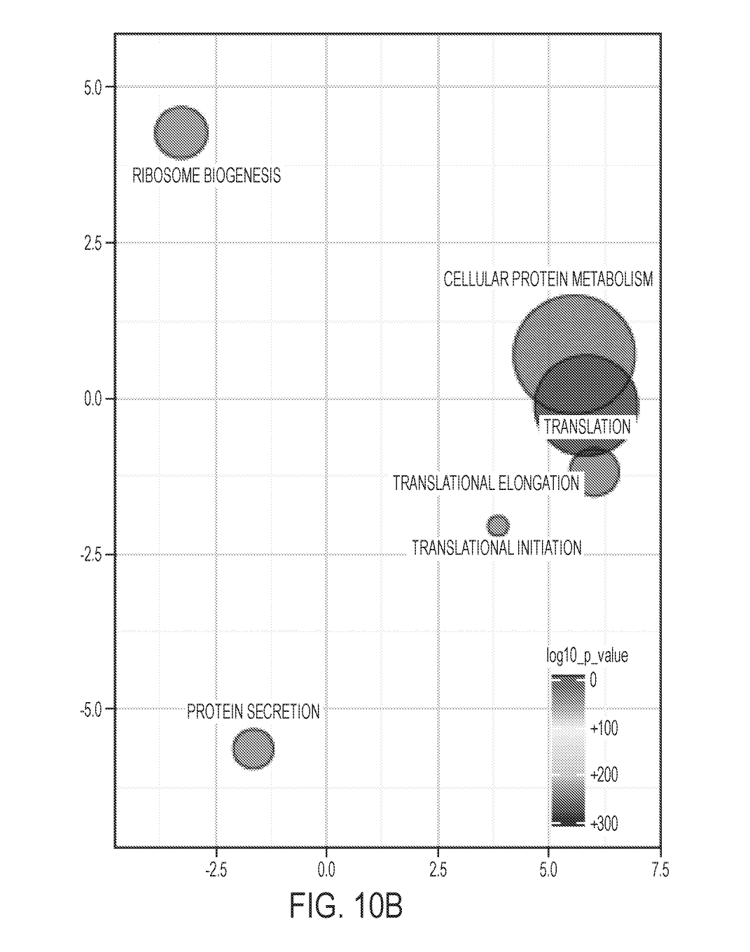

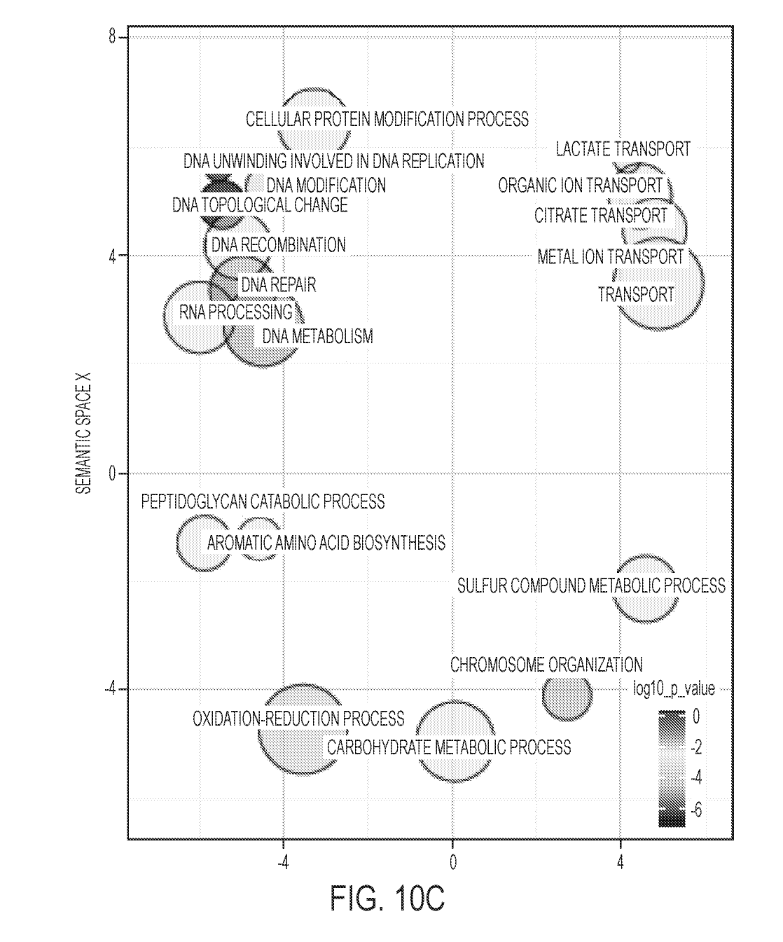

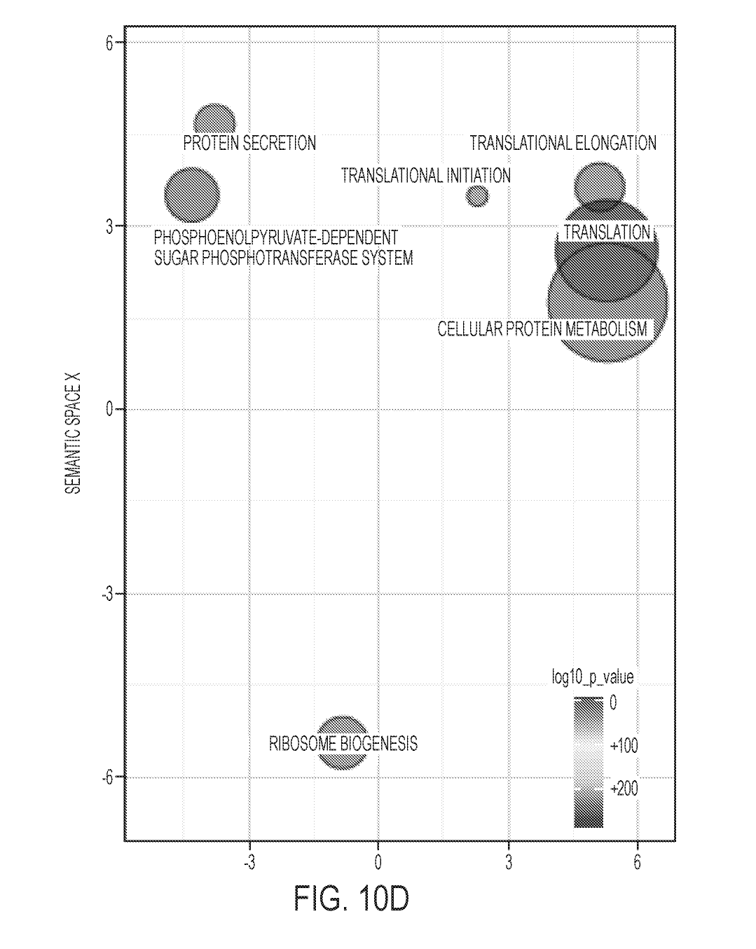

In order to identify activities that could be related to the initial steps of periodontal disease progression community-wide expression profiles of samples at baseline from sites that did not progress vs. sites that did. The analysis of Gene Ontology (GO) enrichment terms showed an over-representation in the baseline of progressing sites of terms related to cell motility, transport (iron ion transport, potassium ion transport and amino acid transport), lipid A and peptidoglycan biosynthesis as well as synthesis of aromatic compounds (FIGS. 9A, 10A and 10C). On the other hand in the baseline samples from non-progressing sites there was an over-representation of GO terms related to tricarboxylic acid cycle, metal ion transport, phosphoenolpyruvate-dependent sugar phosphotransferase system and protein secretion (FIGS. 9B, 10B and 10D).

When compared baselines with healthy sites it was found that the clinically healthy sites at baseline from diseased individuals were already impacted by disease. Both baselines from periodontitis progressing and non-progressing sites had an over-representation of GO terms associated with citrate, organic ion and lactate transport, as well as sulfur compound metabolic processes and peptidoglycan catabolism (FIGS. 10A-10D).

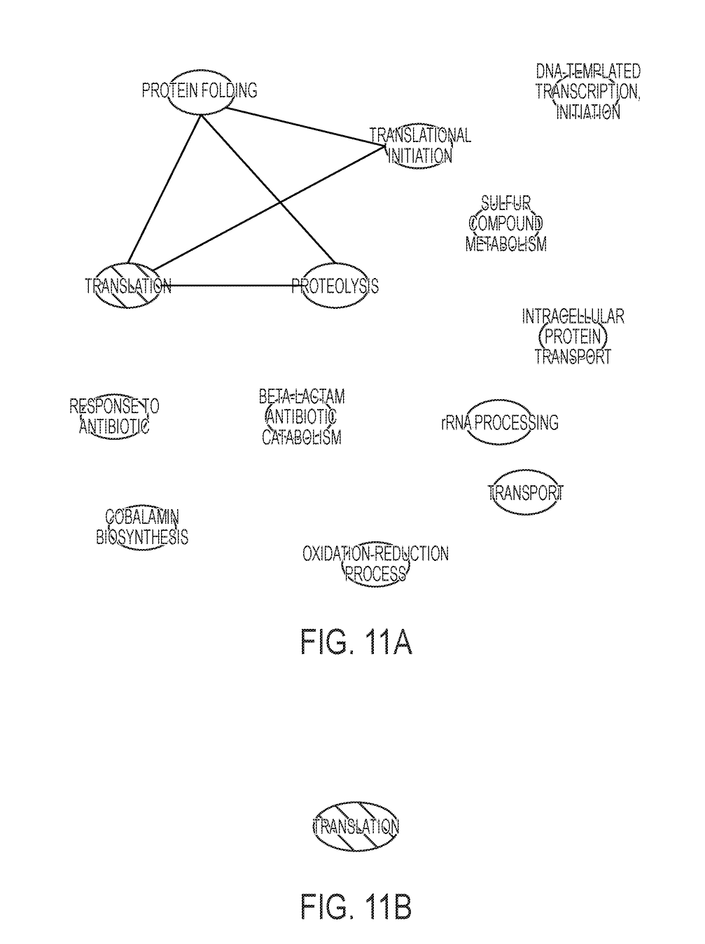

Differences in gene expression of the red complex between baseline from active vs. non-progressing sites were minimal. Gene Ontology (GO) assignment of the differentially expressed genes showed association with sodium ion transport and protein secretion in up-regulated genes and glycine catabolism, intracellular protein transport an response to redox state in down-regulated genes (FIGS. 11A and 11B). P. gingivalis actively up-regulated putative virulence factors (34 in total) while T. denticola with 3 and T. forsythia with 1 up-regulated putative virulence factors seem not specially active at this stage (Table 5).

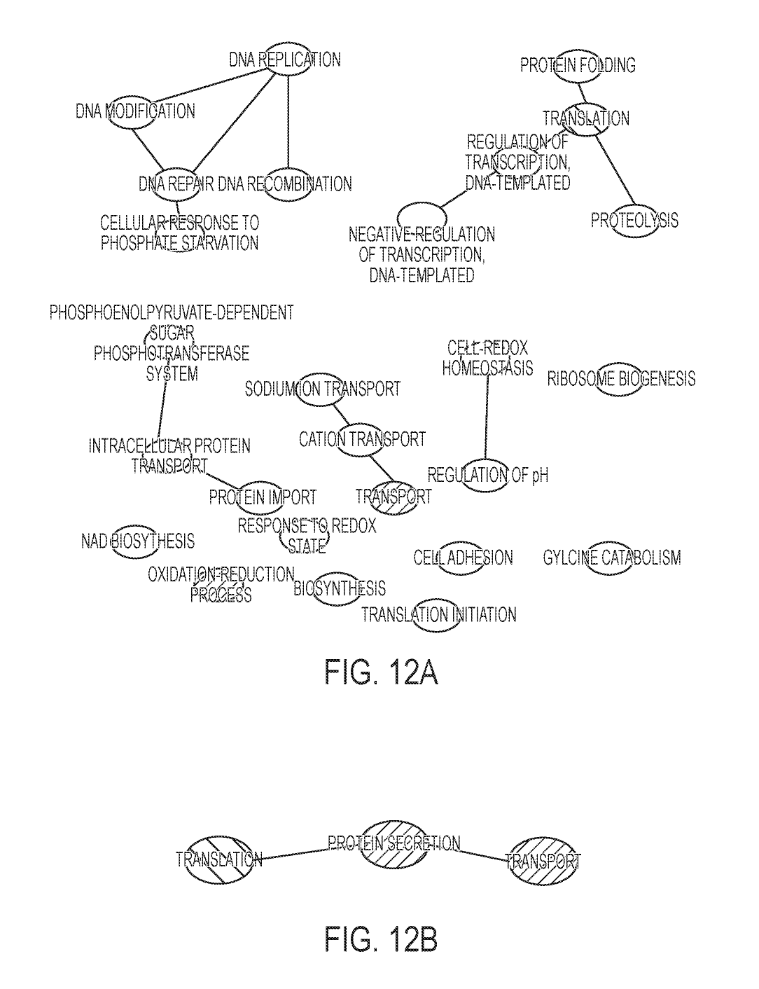

A more complex picture emerged when the behavior of the orange complex was analyzed (FIGS. 12A and 12B). As a whole the members of the orange complex (P. intermedia, P. nigrescens, P. micra, F. nucleatum, F. periodonticum, C. gracilis, C. rectus, S. constellatus, E. nodatum and C. showae) showed up-regulation of genes associated with proteolysis, sodium ion transport, cellular response to phosphate starvation and regulation of pH (FIG. 12A).

### Example 4

### Expression of Putative Virulence Factors in the Oral Community during Periodontitis Progression and at Baseline of Progressing Vs. Baseline of Non-Progressing Sites

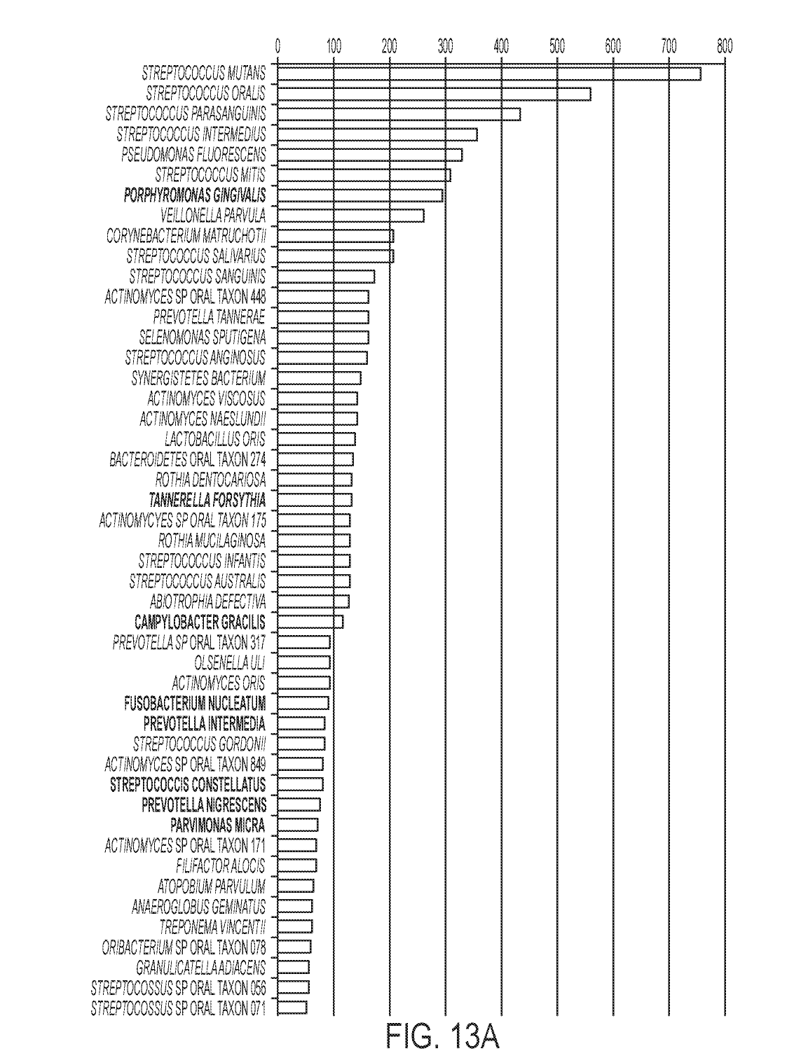

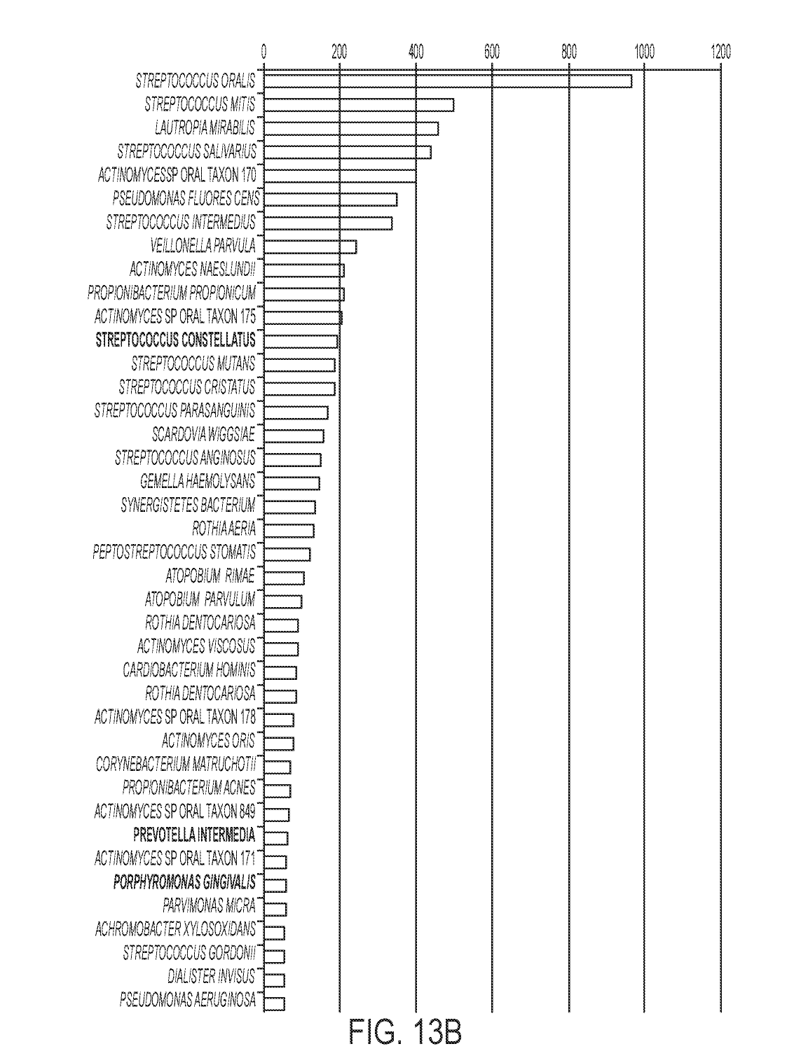

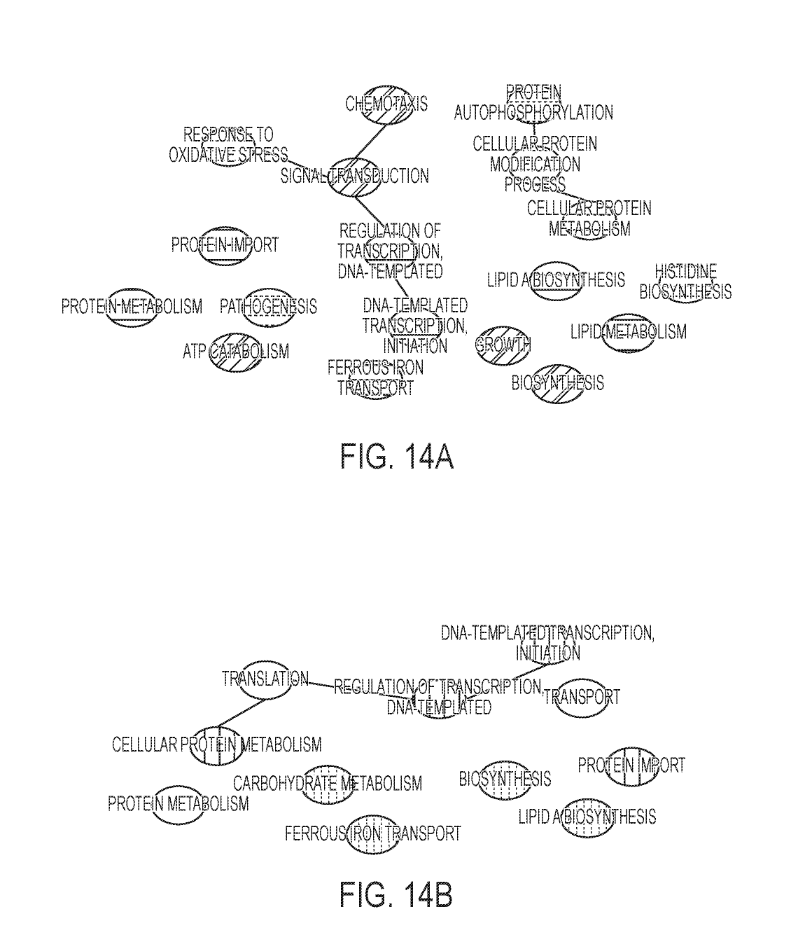

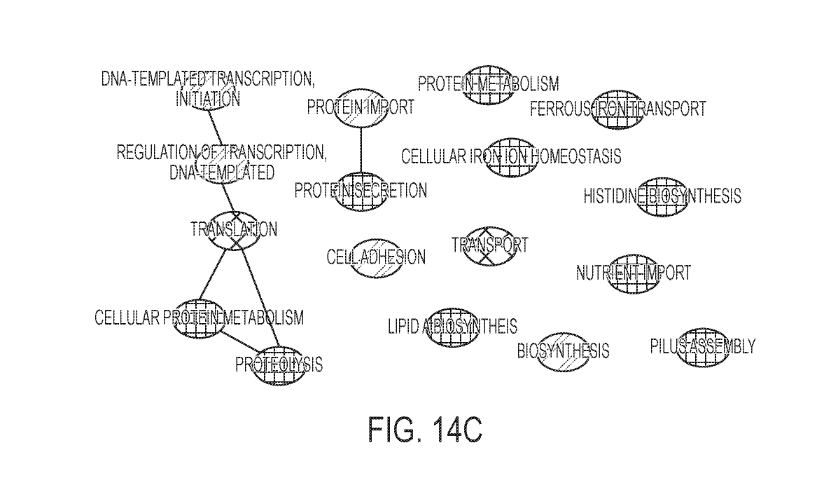

In the case of progressing sites, when comparing baseline vs. break down a total of 9,147 hits of putative virulence factors from 207 species were identified in the genes overrepresented in progressing samples. Nonetheless, not all of them expressed a large number of them, only 47 showed up-regulation of 50 or more putative virulence factors under these conditions. Two members of the red complex P. gingivalis and T. forsythia up-regulated a large number of the putative virulence factors up-regulated in the progressing samples (FIG. 13A). More active in these samples were members of the orange complex C. gracilis, P. intermedia, S. constellatus, F. nucleatum, P. nigrescens and P. micra (FIG. 13A). Three members of this complex were especially active in the up-regulation of putative virulence factors: C. gracilis, F. nucleatum and P. intermedia up-regulated respectively 114, 90 and 82 genes that have homology with virulence factors in the applicant's database (Table 5). P. micra upregulated 69 putative virulence factors, P. nigrescens 73, S. constellatus 79, C. rectus 13 and C. showae 11. No putative virulence factor up-regulated from E. nodatum or from F. periodonticum was identified. When looking at the global activities associated with the expression of these virulence factors an up-regulation of genes related to pathogenesis and iron transport, and lipid A biosynthesis based on Gene Ontology (GO) terms was observed (FIG. 14A). Focusing on the members of the red complex an up-regulation of genes associated with iron transport and lipid A biosynthesis could also be seen (FIG. 14B). More interestingly were the results associated with the orange complex. Adding to the same activities mentioned for the red complex, members of the orange complex up-regulated genes involved in cell adhesion, proteolysis and pilus assembly during periodontitis progression (FIG. 14C).

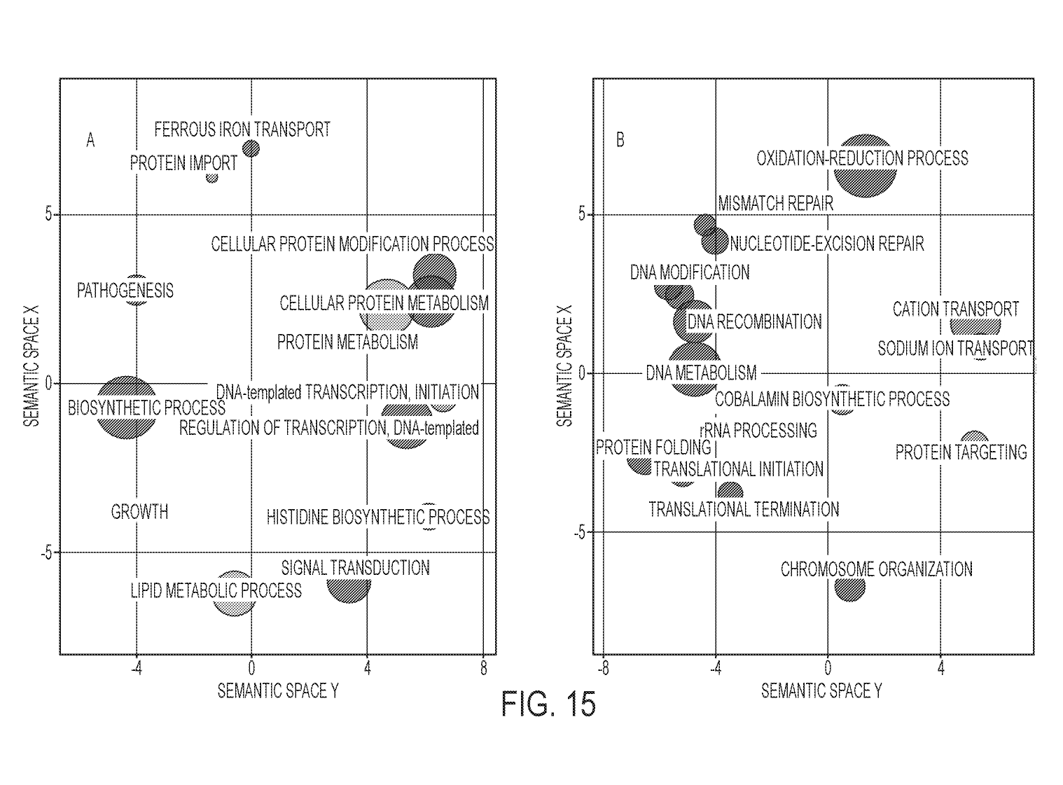

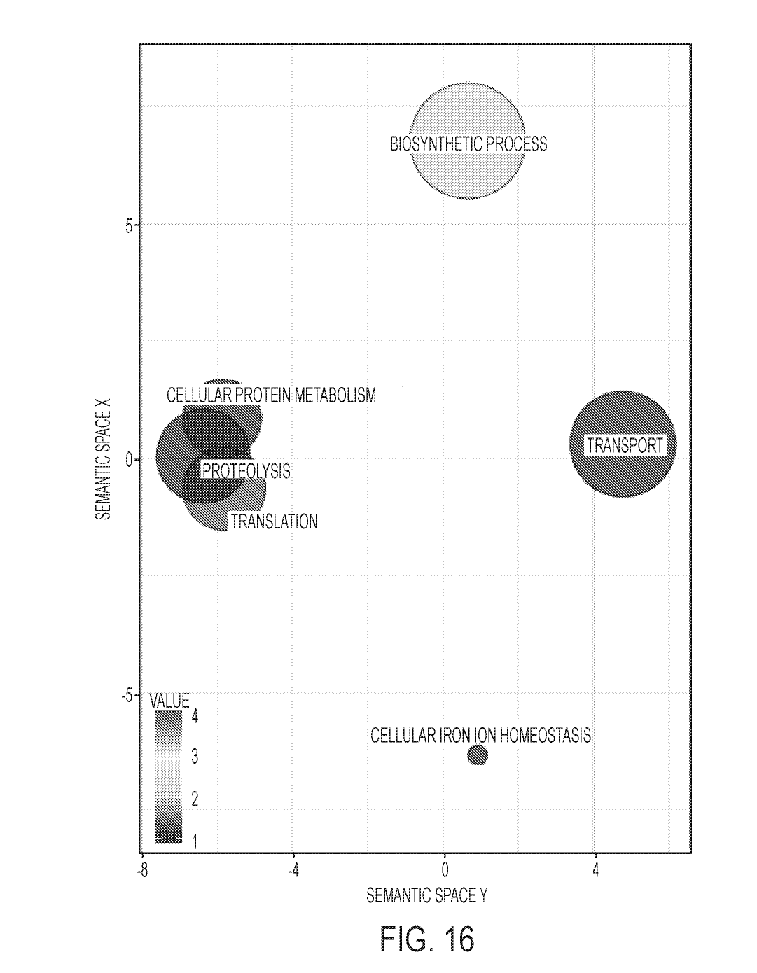

The global activities associated with the expression of virulence factors comparing baselines of non-progressing vs. periodontitis progressing sites were examined and it was found that in the baseline of progressing sites the major activities corresponded to pathogenesis and ferrous ion transport while in non-progressing baselines there was an over-representation of GO terms related to cobalamin biosynthesis and sodium ion transport (FIGS. 15A and B). Comparing the expression of virulence factors at baseline gave a better understanding of the role of two complexes (red and orange in the first stages of disease. While the red complex did not show significant over-representation of any GO terms, members of the orange complex were already up-regulating genes involved in proteolysis and iron homeostasis (FIG. 16). Nonetheless, surprisingly enough, a set of organisms was identified that were highly active transcribing genes of putative virulence factors that had not been usually associated with disease. S. oralis, S. mutans, S. intermedius, S. mitis, V. parvula and P. fluorenscens were up-regulating a large number of putative virulence factors in both analysis (baseline vs. periodontal disease progression, comparing baseline in periodontal disease progression with baseline in non-progressing sites, FIGS. 13A and 13B).

All of the above cited organisms up-regulated different oligopeptide transport systems (oppA, oppD, oppF and oppB). S. oralis, S. mutans, S. intermedius, S. mitis, P. fluorenscens, and V. parvula up-regulated several hemolysins, manganese ATP-binding cassette (ABC) transporters, manganese superoxide dismutase, and a protein serine threonine phosphatase (PrpC) involved in regulation of stationary phase (Table 5). S. oralis and V. parvula up-regulated vitamin B12 ABC transporters (Table 5). S. oralis, S. mutans, S. intermedius, S. mitis, and P. fluorenscens all up-regulated Clp protease and LytR transcriptional attenuator. P. fluorescence up-regulated all genes associated with flagellar synthesis (flaA, flat, fleN, flgA, flgC, flgF, flgI, flgJ, flgK, flgN, flhA, flhB, flhF, fliD, fliF, fliG, flil, fliK, fliN, fliP, fliQ, fliR, fliS and motB) and genes related to chemotaxis (Table 5).

In spite of the commonalities in up-regulated genes when comparing baselines of progressing to non-progressing sites and progression, there were also specific signatures of the two comparisons. For instance, S. oralis and S. mitis up-regulated collagen adhesion proteins and V. parvula TonB dependent receptors when comparing baselines but not during progression.

### Example 5

### Integrating Expression Profiles and Clinical Traits during Periodontitis Progression

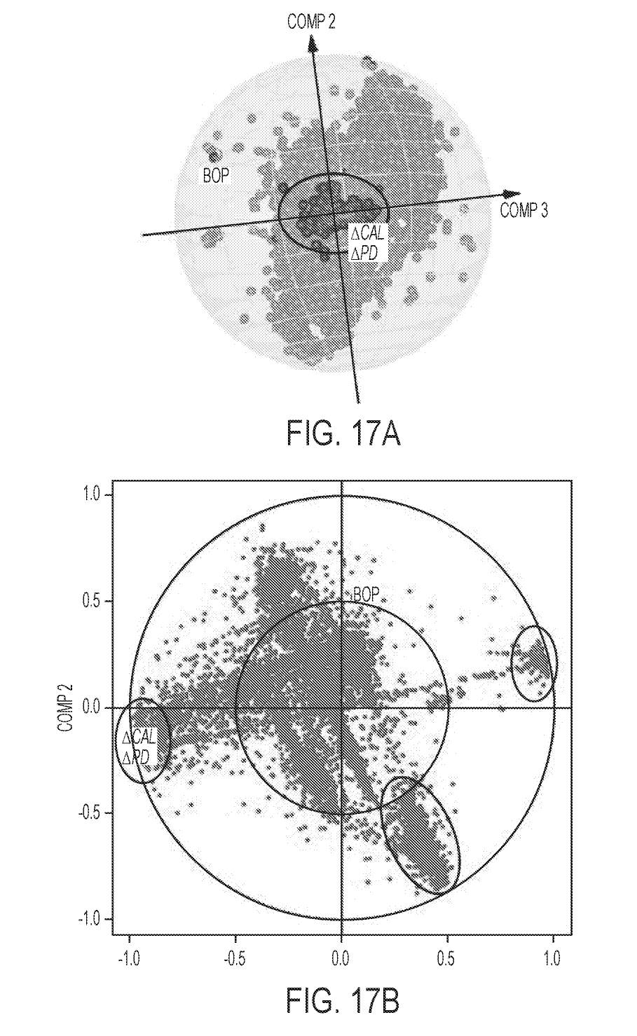

Integrating microbiological functions with clinical parameters is still one of the challenges in omics analysis. Multivariate statistical analysis and visualization tools implemented in the R package mixOmics (R package mixOmics provides statistical integrative techniques and variants to analyze highly dimensional data sets, González et al., BioData Min.; 5:19, 2012) were used to identify relevant association between gene expression and the clinical traits: bleeding on probing (BOP), increase in pocket depth (ΔPD) and increase in clinical attachment level (ΔCAL). The sparse Partial Least Square (sPLS) correlations were calculated between the clinical traits and both active species (RNA levels of expression normalized by metagenome abundance) and profiles of gene expression in the periodontitis progressing sites. FIGS. 17A and 17B show the visualization of those relationships using Correlation Circle Plots. Not surprisingly, ΔPD and ΔCAL were highly correlated and belonged to the same principal component. There were three sets of genes that were highly correlated with 3 principal components (FIG. 17B), 1 of them correlated with the periodontitis progression of the clinical parameters studied. There were no genes whose 279 profiles correlated with BOP (FIGS. 17A and 17B). Interestingly, 2 large set of genes correlated with other components (FIG. 17B), but which possibly corresponded to another clinical trait not analyzed in this study.

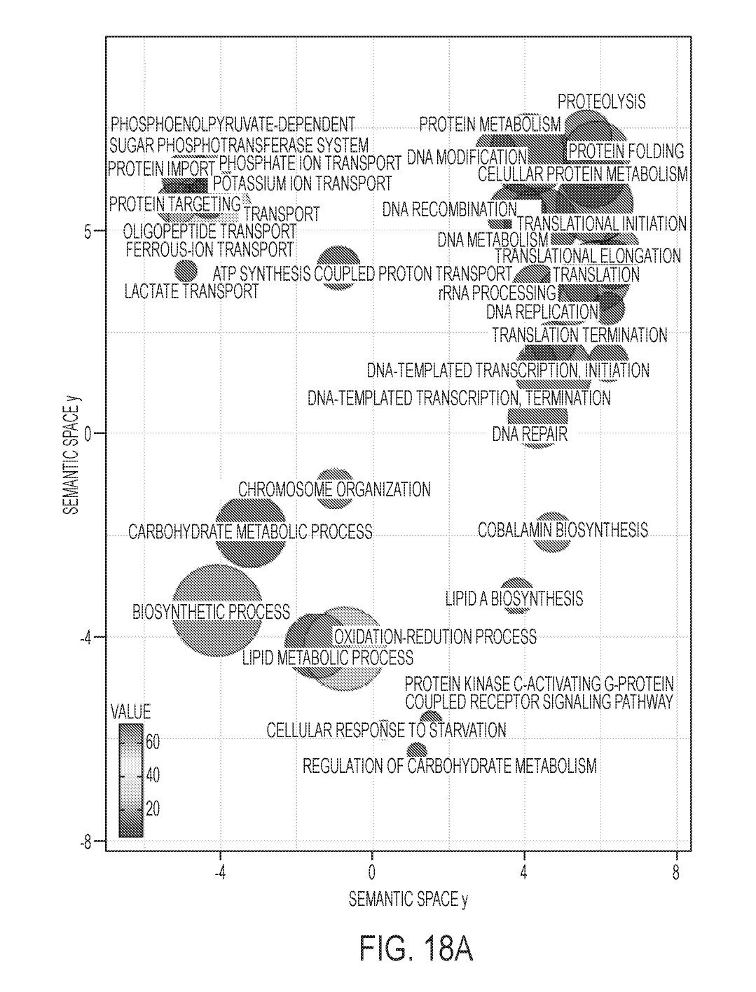

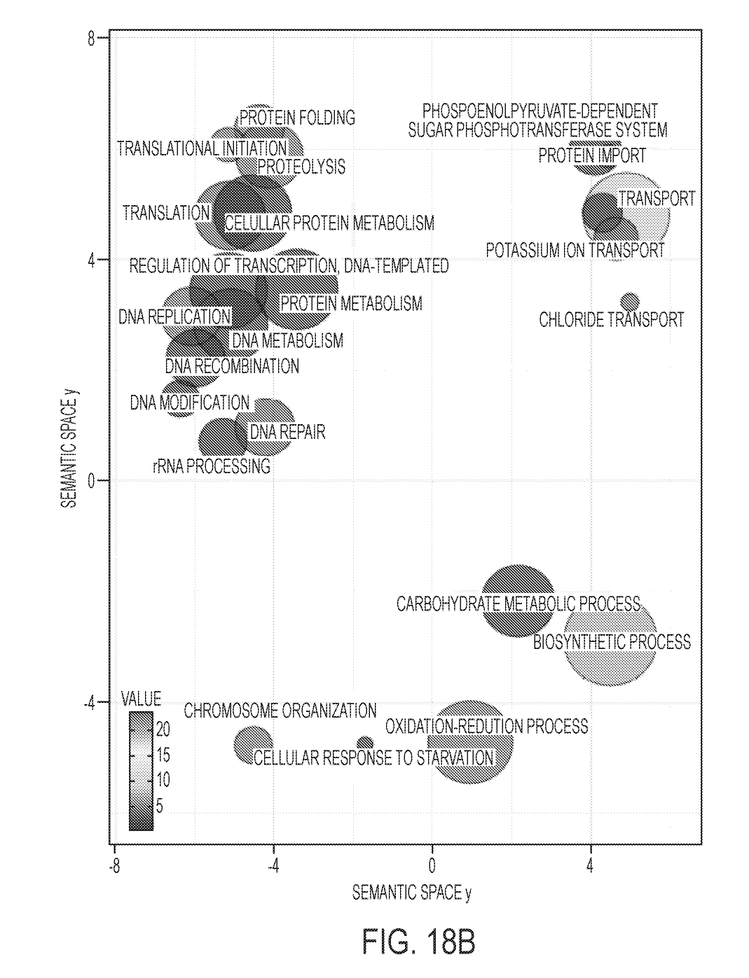

Correlation structures between clinical traits and species and gene expression were also analyzed using Relevance Networks (Gonzalez et al., BioData Min.; 5:19, 2012). This method generated a graph where nodes represent variables and the edges represent the correlations. The correlation of gene expression profiles gave a large number of genes that correlated with the clinical parameter profiles. Not surprisingly, BOP, which is a discrete variable, did not correlate with any gene profiles. ΔPD and ΔCAL correlated with a large number of gene profiles, even with an r=0.95. GO terms were then assigned to the correlated genes and these results were summarized using reduce and visual Gene Ontology (REVIGO) method. Specific patterns of activities associated with increases in PD and CAL were detected (FIGS. 18A and 18B). Among those patterns it was found that the profiles of expression of phosphoenolpyruvate-dependent sugar phosphotransferase system, proteolysis and potassium transport were associated with the worsening of those 2 clinical parameters.

### Example 6

### Viral Activity in the Oral Cavity

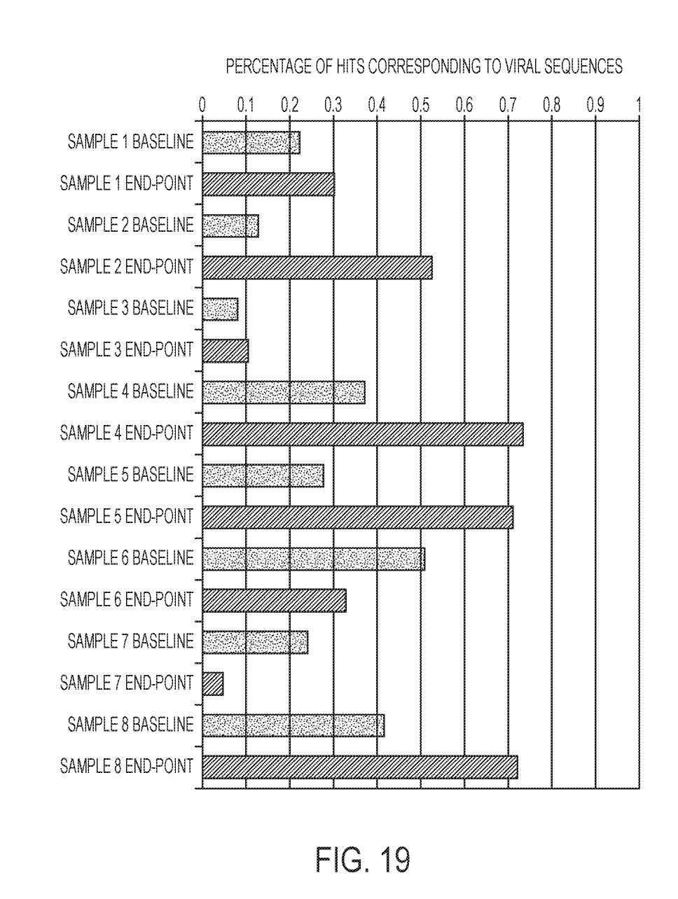

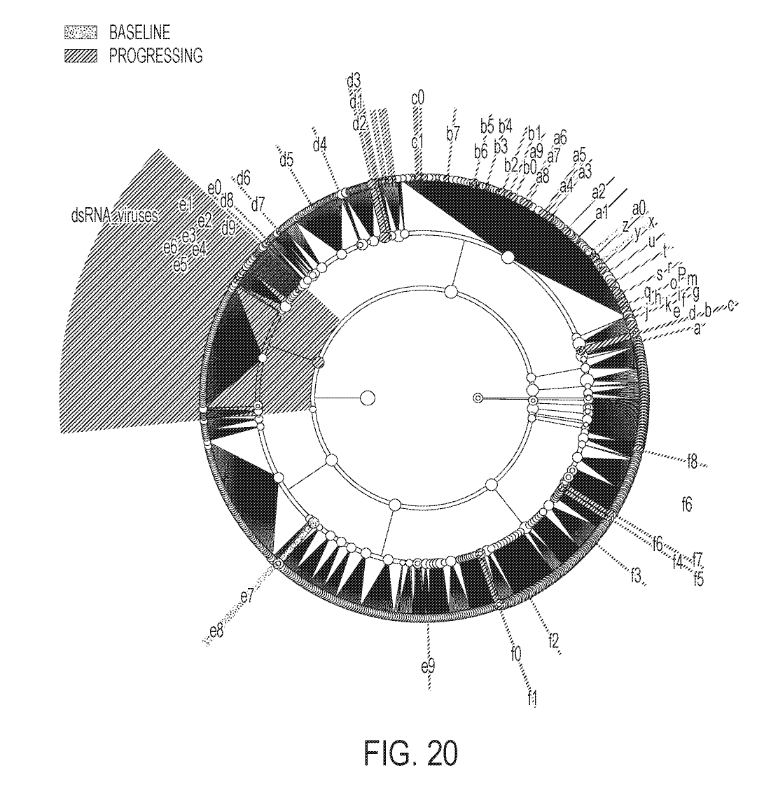

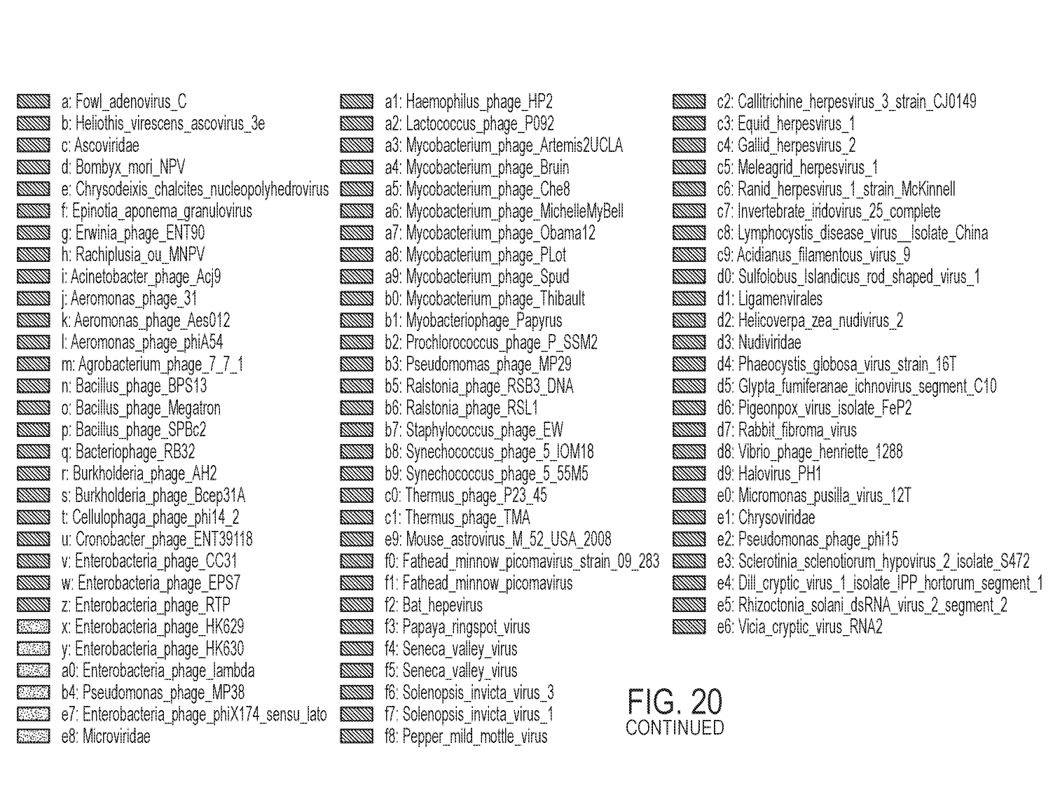

The presence of eukaryotic viruses and bacteriophages was also examined, which have been previously associated with disease (Slots et al., Periodontol 2000, 53:89-110, 2010) and may play a role in shaping the bacterial community (Pride et al., ISME J, 6:915-926, 2012). Viral activities were identified in all samples although the number of transcripts represented a small fraction of all hits identified, between 0.04 and 0.7% of all hits were of viral origin (FIG. 19). To confirm that those hits really belong to viral sequences consensus sequences were obtained from the bam files of our alignments and BLASTed against the nr database at NCBI. Consensus sequence from 14 out of the 16 samples analyzed had sequences with significant matches to viral sequences. When comparing the relative activities of viruses at baseline and periodontitis progression, high activity of phages and herpesvirus was observed in the progressing sites in relation with the baseline samples (FIG. 20).

A combined metagenomic/metatranscriptomic approach was recently used to characterize the functional dysbiotic phenotype of the oral microbiome during early stages of periodontitis progression. In a recent report it was taken advantage of Next Generation Sequencing (NGS) techniques and the fact that a large number of genomes from oral isolates have been sequenced (Chen et al., Database J Biol Databases Curation. 2010:baq013, 2010) to infer functional differences between the subgingival microbiota of periodontal health and chronic severe periodontitis (Duran-Pinedo et al., ISME J. 8:1659-72, 2014).

Recent studies on community composition that used NGS analysis compared healthy samples with chronic periodontitis concluding that members of the genus Prevotella, Fusobacterium, Treponema, Sinergistites, Filifactor, and Porphyromonas and candidate division TM7 were more abundant whereas Actinomyces and Streptococcus were less abundant in samples from periodontitis compared to healthy subjects. These results agreed with the associations of red and orange complexes with periodontal disease previously postulated using other detection methods (Socransky et al., Periodontol 2000. 38:135-87, 2005; Haffajee et al., Oral Microbiol Immunol. 23:196-205, 2008). Similar results were observed in a previous study by the applicants when comparing health and chronic periodontitis (Duran-Pinedo et al., ISME J. 8:1659-72, 2014). Although these analysis were performed using 16S rDNA sequencing while the applicants' analysis was metagenomic analysis it was also found that in the periodontitis progressing sites Fusobacterium and Prevotella were more abundant when the teeth broke down than at baseline while the opposite was true for the genus Streptococcus. Non-progressing sites had almost the same metagenomic composition at baseline and at the time point when the sample was taken.

Examining baseline samples might give insights on the changes in microbial composition and activities that define the initial stages of periodontital disease progression. Large differences were observed when comparing baseline samples of periodontitis progressing with non-progressing sites. Members of the genera Porphyromonas, Treponema, Tannerella and Prevotella, among others, were more abundant at baseline on sites that progressed. Members of the genus Streptococcus were more abundant at baseline in non-progressing sites.

Differences in the phylogenetic assignment of active members of the microbial community were also examined. It was found that, as in the metagenome, no major differences were observed in non-progressing sites when comparing baseline and final time point. However, the differences were profound in the active sites and when periodontitis progressing sites at baseline and final time point were compared and even more profound when the 2 baselines were compared. During periodontitis progression the profile was similar to what was observed in a previous report. P. gingivalis, T. denticola and T. forsythia were highly active during progression while Streptococcus species (sp.) were highly active at baseline. Interestingly, Synergistites species (sp.) and the archaea Methanobrevibacter species (sp.) were more active in progression and had been previously associated with periodontal disease.

When looking at differences at baseline between active and non-progressing sites it was found that known periodontal pathogens T. denticola and T. forsythia were not significantly more active while P. gingivalis was. In contrast, F. alocis, E. nodatum and several Prevotella species (spp.) were more active in the sites that would progress than in the site that would remain non-progressing. Both F. alocis and E. nodatum, a member of the orange complex, have been previously associated with periodontal disease.

Testing for differential representation of Gene Ontology (GO) terms gives an overall view of the metabolic activities of the whole community under different environmental conditions. Interestingly, when the expression profiles of baseline and non-progressing sites (that did not change based on clinical parameters) were compared no gene was identified as differentially expressed, which indicated that the community as a whole did not change its expression patterns during the period passed between the first visit and the time where samples were taken.

When looking at periodontal disease progression the results pointed to several functional signatures characteristic of the active sites. At the breakdown point active sites were actively expressing genes associated with oligopeptide and ferrous iron transport. Oxidative stress is one of the consequences of the host inflammatory response to the microbial challenge and bacteria must act to defend themselves against this host defense mechanism, which is probably accentuated with the progression of disease. Iron is an essential enzymatic cofactor and the in situ over-expression of genes related to its transport in the microbial community during severe chronic periodontitis was already shown. At baseline, GO terms associated with isoprenoid and polysaccharide biosynthesis, sulfur compound metabolic processes, potassium ion transport and protein kinase C-activating protein coupled receptor signaling pathway were highly enriched. Lipopolysaccharide (LPS) is a key factor in the development of periodontitis and high levels of lipopolysaccharide (LPS) from P. gingivalis have been reported to delay neutrophil apoptosis and provide a mechanism to modulate the restoration and maintenance of inflammation in periodontal tissues. Hydrogen sulfide production from amino acids and peptides has been reported in periodontal bacteria and the different efficiency of use of these compounds could be important determinants of the periodontal microbial ecology. More puzzling is the over-representation of GO terms related to potassium transport. Potassium transport systems have been associated with pathogenesis in other organisms such as Staphylococcus aureus and Salmonella, but not in oral bacteria. Although significant higher levels of potassium in the gingival crevicular fluid (GCF) and saliva have been reported in periodontitis.

Comparing baseline metabolic activities of non-progressing vs. periodontitis progressing sites will give a better understanding of the initial stages of disease and the role that the microbial community plays at this early stage of pathogenesis. Among those functional signatures were found: citrate transport, iron transport, potassium transport, amino-acid transport, isoprenoid biosynthesis and ciliary and flagellar motility. Citrate transport has been linked to iron transport and virulence in other organisms, such as Shigella and Pseudomonas. This was in accordance with previous observations in severe chronic periodontitis sites (Duran-Pinedo et al., ISME J, 8:1659-1672, 2014). As mentioned above, the efficiency in utilizing various amino acids and peptides is among the key determinants of the periodontal microbial ecology and its uptake might give additional advantages to certain members of the microbial community. In the active sites, there seemed to be a shift from amino-acid uptake to oligopeptide uptake throughout the breakdown process. Isoprenoid biosynthesis, probably involved in the synthesis of peptidoglycan, was also over represented in active sites. Isoprenoids are a large, diverse class of naturally occurring organic chemicals which are essential for cell survival. The 2C-methyl-D-erythritol 4-phosphate (MEP) pathway has been implicated in the virulence of Listeria monocytogenes, Mycobacterium tuberculosis, Brucella abortus and evidence indicates that the MEP pathway may be involved in intracellular survival by combating oxidative stress. Moreover, a metagenomic analysis of the human distal gut microbiome revealed that MEP pathway genes are highly abundant in that community; perhaps reflecting the abundance of the MEP pathway in bacteria in general. Finally, ciliary and flagellar motility as well as chemotaxis genes that could direct bacterial movement were all part of the signature activities at the initial stages of progression. Motile pathogenic members of the oral community, such as Treponema spp. (species), possess the capacity for tissue invasion thanks to the synthesis of flagella (Heinzerling et al., Infect Immun, 65:2041-2051, 1997; Lux et al., Infect Immun, 69:6276-6283, 2001); these results indicate that this fraction of the community is already active at the initial stages of progression.

Historically, members of the red and orange complexes have been associated with chronic periodontitis. Consistent with their postulated role in periodontitis progressing sites a high level of expression of putative virulence factors being expressed by members of both complexes when breakdown occurs was observed. However, at the baseline of the present studies it seemed that the relative importance of these complexes in the active sites was reduced. Only P. gingivalis, S. constellatus, and P. intermedia were actively expressing putative virulence factors.

Interestingly, members of the red complex showed enrichment in response to antibiotics (beta lactamase activity) during periodontal disease progression and even at baseline of progressing sites. The same phenomenon was observed at whole community-level in a previous study comparing healthy sites vs. chronic severe periodontitis (Duran-Pinedo et al., ISME J. 8:1659-72, 2014). Beta-lactamase activity had been observed in adult periodontitis at low-level enzymatic activity but with high prevalence and seemed to be a frequent phenomenon in samples from polymicrobial diseases . It is still unknown what role this enzymatic activity plays on the progression of the disease given that the patients of this study were not treated with antibiotics at the time of sampling.

CRISPR-associated genes in P. gingivalis, T. forsythia, C. gracilis, C. rectus, C. showae, P. nigrescens and S. constellatus were highly up-regulated during periodontitis progression. Phage activity was observed in all samples analyzed, which could explain the high level of production of CRISPR associated proteins as a mechanism of defense against viral activity . However, it is possible that CRISPR-associated proteins are playing a broader role in the virulence mechanisms of periodontitis. Thus, recently CRISPR-Cas systems have been linked to stress responses and virulence in bacteria and to competitive interactions between members of the red complex.

P. gingivalis, P. nigrescens and P. intermedia up-regulated all the traA-Q and mob genes in their chromosomal conjugative transposons. These genes are required for formation of a conjugal pore and DNA mobilization The up-regulation of these genes indicated conjugative transposons mobilization in Porphyromonas and Prevotella which would agree with evidence of natural horizontal transfer of antibiotic resistance through conjugative transposon mobilization in those organisms However, the mobilization of those conjugative transposons was most likely driven by the presence of antibiotics. In the current study, subjects did not use systemic antibiotics during the monitoring period when samples were collected. Therefore, it is not clear what signal(s) triggered this mobilization of conjugative transposons. It is noteworthy that this phenomenon was not observed in severe chronic periodontitis samples.

The idea that the whole community acts as pathogen rather than specific organisms has been gaining traction in recent years. In agreement with this hypotheses it was found that a group of organism not usually considered pathogens were up-regulating a large number of putative virulence factors in active sites. Among these groups, it was observed that some streptococci, including S. mitis and S. intermedius, were especially active. Although S. mitis and S. intermedius are usually associated with periodontal health, they have also been found to form part of the community in periodontitis. V. parvula was found to be highly active in both progressing and baseline sites, which was surprising since V. parvula is mostly associated with a healthy community. However, streptococci and V. parvula have been identified as part of a cluster associated with periodontitis in adolescents. Another surprising finding was the identification of P. fluorescens as one of the top producers of virulence factors. This is not an organism usually associated with periodontitis, although another member of its genus, P. aeruginosa, has indeed been associated with other important pathologies such as cystic fibrosis. In our previous study on chronic severe periodontitis, a similar behavior was also observed where the whole community, and not only known periodontal pathogens, expressed more putative virulence factors in diseased sites. Among the most active producers of putative virulence factors was Corynebacterium matruchotii (Duran-Pinedo et al., ISME J. 8:1659-72, 2014), which has also been associated with periodontitis in microbiome studies. Interestingly, it was also found that this organism is highly active in periodontitis progressing sites but not at the baseline, indicating a shift into a ‘pathogenic microbial community’.

Then an association was established between profiles of clinical parameters such as BOP, ΔPD and ΔCAL with profiles of gene expression. BOP showed no association with changes in gene expression profiles. This is not surprising since BOP is a discrete variable. Nonetheless, ΔPD and ΔCAL were highly associated with proteolytic activity and potassium ion transport. Additionally, ΔPD was associated with cobalamin biosynthesis and ferrous and oligopeptide transport. Proteolysis has been recognized as an important virulence determinant in periodontitis progression. In the case of vitamin B12 (cobalamin) synthesis, an up-regulation of the vitamin B12 ATP-binding cassette (ABC) transporter btuFCD system in P. gingivalis and T. forsythia and some members of the orange complex was observed. P. gingivalis harbors all the genes necessary to convert precorrin-2 into cobalamin, but it lacks the genes for the synthesis of precorrin-2. In a previous study on chronic severe periodontitis an up-regulation of btuFCD system in P. gingivalis and T. forsythia was also observed (Duran-Pinedo et al., ISME J. 8:1659-72, 2014). An increase in synthesis and release to the external medium by other organisms of cobalamin might give members of the red and orange complex an ecological advantage if they start scavenging it.

The initial causes for transition from a healthy microbial community to a dysbiotic one are still not well understood in great part due to the complexity of the oral community. Using a metagenomic/metatranscriptomic approach, and comparing baseline samples from the same individuals, the study of the physiological changes in the microbial community that are associated with the initial stages of dysbiosis had begun. Here, it was shown that in periodontitis progression there are certain characteristic activities that were associated with the onset of breakdown in specific teeth. Among those urea metabolism, citrate transport, iron ion transport, potassium ion transport, amino-acid transport, isoprenoid biosynthesis and ciliary and flagellar motility were found to be signatures of the initiation of periodontitis progression.

The data presented here indicate that regardless of the overall composition of the community, certain metabolic signatures are consistent with disease and progression. For instance, the community composition in the progressing active sites was relatively different from the composition of the community in chronic severe periodontitis sites previously described (Duran-Pinedo et al., ISME J. 8:1659-72, 2014). Nonetheless, in both cases it was observed that iron and oligopeptide transport activities were highly associated with advance stages of disease. Moreover, as shown in a previous study on chronic severe periodontitis (Duran-Pinedo et al., ISME J. 8:1659-72, 2014), the present results show that the whole community and not just a handful of oral pathogens was responsible for an increase in virulence that could lead to periodontitis progression. Finally, it was found that certain ecological changes could explain the evolution of certain clinical parameters. As discussed in the previous section an increase in production of cobalamin could exacerbate the growth of periodontal pathogens that lack the capabilities to synthesis this compound and explain, at least in part, the association of these organisms with increase in disease severity.

### Example 8

### Cobalamin Synthesis Inhibitors are Useful in Breaking up Plaque

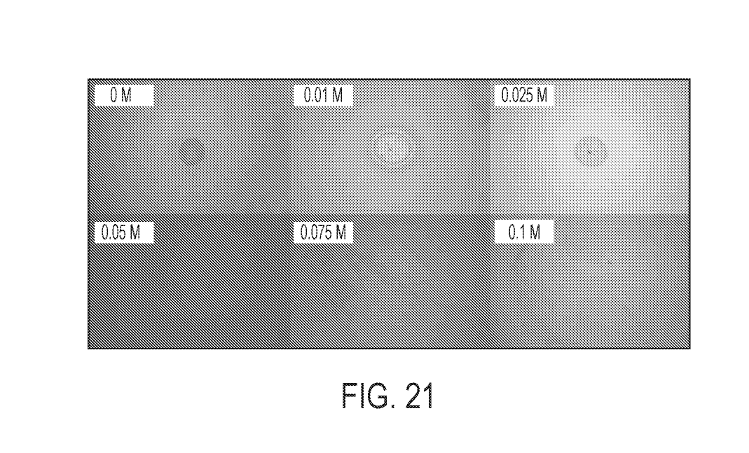

Dental plaque is a biofilm that builds up on teeth and contains bacteria associated with cavities, gingivitis, and periodontitis. Plaque samples were obtained from four human subjects. Plaque bacteria from each subject was plated on blood agar and cultured in the presence or absence of various concentrations of Sodium tripolyphospate (TPP), which inhibits Cob(I)alamin adenosyltransferase. Interestingly, the cobalamin synthesis inhibitor, TPP, completely inhibited oral plaque growth in a dose dependent manner. These results indicate that cobalamin inhibitors are useful in breaking up biofilms that form on dental surfaces. FIG. 21 shows representative results from one subject.

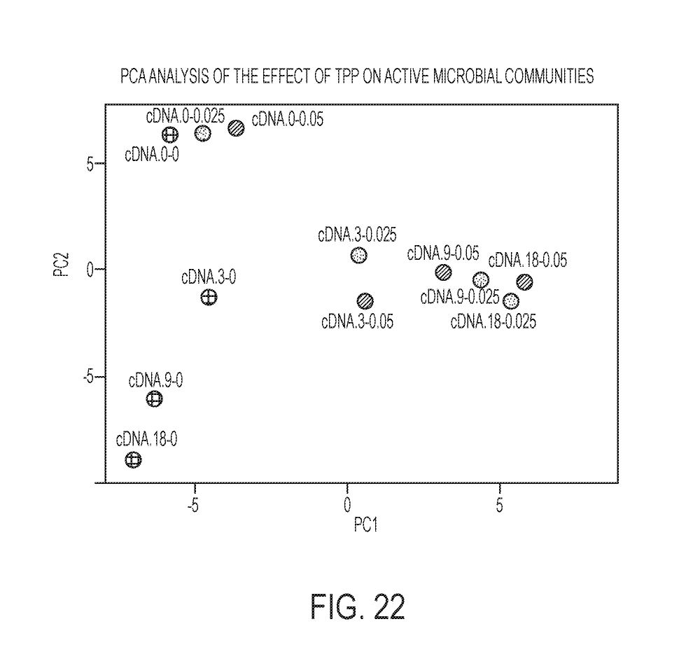

Principal component analysis was used to analyze changes in oral microbial community composition. Results of an analysis of the active community composition isolated from a human subject was carried out by analysis of 16S rRNA genes from cultures of plaque. The panel of species identified included 500 different species. Samples from the culture were taken at 0, 3, 9 and 18 hours. No change in the composition of bacteria was seen in the absence of TPP. Interestingly, the presence of TPP affected all of the bacterial communities in similar ways at all concentrations as shown (FIG. 22).

### Example 9

### TPP Inhibited the Growth of Streptococcus sanguinis

Oral plaque containing Streptococcus sanguinis was obtained from a human subject and cultured in liquid culture in the presence or absence of TPP. S. sanguinis is a gram positive bacteria that binds to the surface if the teeth, where it acts as a tether for the attachment of other bacteria that form dental plaque, and contributes to the development of caries and periodontal disease. S. Sangunis has all of the genes required for anaerobic cobalamin biosynthesis.

Microbes exist in a range of metabolic states (e.g., dormant, active and growing) and analysis of ribosomal RNA (rRNA) is frequently employed to identify the ‘active’ fraction of microbes in samples. In our analysis we observed that the fraction of active S. Sanguinis after three hours of incubation in the presence of 25 mM and 50 mM TPP was significantly lower than in our control with no TPP added. This is of particular interest given that other bacterial species may utilize and or depend on the cobalamin synthesized by S. Sanguinis, thus making this species a keystone esential to maintain a mature biofilm.

Other bacteria whose activity was significantly reduced in the presence of TPP at concentrations as low as 25 mM include the following bacteria: S. intermedius, S. Genus probe 1, Solobacterium moorei, and Rothia dentocariosa.

The experiments above were performed with the following methods and materials.

### Power Calculation

In order to assess the sample size required the R package RNASeqPower was used, an open source software by BIOCONDUCTOR© to calculate sample size from RNA-sequencing (Hart et al., J Comput Biol.; 20:970-8, 2013). The average coverage was first estimated using ‘SAMtools depth’ command from the SAMtools package (Li et al., Bioinforma Oxf Engl.; 25:2078-9, 2009; Sequence Alignment Map (SAM) tools is a generic format for storing large nucleotide sequence alignments and for providing various utilities for manipulating alignments in the SAM format). According to this analysis, with false discovery rate (FDR) of 0.05 and a target effect size of 2, subjects in each group were needed to have a power of 0.9.

### Study Design, Subject Population and Sample Collection

The subjects in the present study were recruited as part of a multi-center clinical trial to determine biomarkers of periodontal disease progression (Clinical Trials.gov ID-NCT01489839). Under this ongoing study, subjects were monitored clinically for a period of up to 1 year every 2 months in order to detect periodontal sites and subjects with periodontal disease progression. Subgingival microbial samples were collected from up to 32 sites per subject per visit. The Institutional Review Board at The Forsyth Institute approved all aspects of the study protocol. The study was described thoroughly to all subjects prior to obtaining informed consent. Inclusion criteria were: study subjects were >24 years of age, had ≥20 natural teeth (excluding third molars), had at least 4 teeth with at least 1 site of pocket depth (PD) of 5 mm or more and concomitant clinical attachment loss (CAL) greater than or equal to 2 mm, and radiographic evidence of mesial or distal alveolar bone loss around at least 2 of the affected teeth, and were in good general health (Table 3). Exclusion criteria: Subjects were excluded if they were current cigarette smokers; were pregnant or nursing; received antibiotic or periodontal therapy in the previous six months; had any systemic condition potentially affecting the course of periodontal disease (e.g. diabetes or AIDS); made chronic use of nonsteroidal anti-inflammatory drugs, or had any condition requiring antibiotic coverage for dental procedures. Periodontal disease progression at a site was defined by an increase in CAL≥2 mm at any follow-up visit compared with baseline. Stable sites were characterized by no change in CAL>1 mm from baseline. 8 stable sites and 8 progressing sites from the 9 subjects were analyzed (Table 4). One stable site and one progressing site were collected, both at baseline and at the end-point of analysis. For 7 of the 9 subjects both periodontitis progressing and stable sites matched the initial baselines. Samples were processed as described below.

TABLE 3
Clinical and demographic characteristics of study subjects

| | | | Mean | % of sites with |
|---|---|---|---|---|
| Subject | Age (years) | Mean PD (mm) | CAL (mm) | PD ≥5 mm |
| 1 | 53 | 2.1 | 1.4 | 7% |
| 2 | 32 | 2.5 | 1.8 | 15% |
| 3 | 54 | 3.0 | 3.0 | 17% |
| 4 | 50 | 3.9 | 3.3 | 41% |
| 5 | 59 | 3.1 | 3.3 | 21% |
| 6 | 42 | 2.1 | 2.5 | 3% |
| 7 | 75 | 1.9 | 3.4 | 10% |
| 8 | 66 | 2.0 | 2.2 | 7% |
| 9 | 63 | 1.9 | 1.0 | 4% |

PD—pocket depth,
CAL—clinical attachment loss.

TABLE 4
Clinical and demographic characteristics of periodontitis
progressing and stable sites

| | | | | | Progression |
|---|---|---|---|---|---|
| Subject | Site* | Visit (months) | PD (mm) | CAL (mm) | (0/1) |
| 1 | 361 | 0 | 3.0 | 2.0 | 1 |
| | 361 | 2 | 5.0 | 4.0 | |
| 1 | 353 | 0 | 2.5 | 1.5 | 0 |
| | 353 | 2 | 3.0 | 2.0 | |
| 2 | 473 | 0 | 3.0 | 2.0 | 1 |
| | 473 | 2 | 5.0 | 4.0 | |
| 2 | 241 | 0 | 3.0 | 1.0 | 0 |
| | 241 | 2 | 3.0 | 2.0 | |
| 3 | 273 | 0 | 4.0 | 4.0 | 1 |
| | 273 | 2 | 8.5 | 8.0 | |
| 3 | 441 | 0 | 4.0 | 3.0 | 0 |
| | 441 | 2 | 3.0 | 2.0 | |
| 4 | 143 | 0 | 6.0 | 5.0 | 1 |
| | 143 | 2 | 8.0 | 7.0 | |
| 4 | 453 | 0 | 4.5 | 3.5 | 0 |
| | 453 | 2 | 5.0 | 4.0 | |
| 5 | 151 | 0 | 3.0 | 3.0 | 1 |
| | 151 | 2 | 5.0 | 5.5 | |
| 5 | 373 | 0 | 3.0 | 3.0 | 0 |
| | 373 | 2 | 3.0 | 3.0 | |
| 6 | 143 | 0 | 2.0 | 2.0 | 1 |
| | 143 | 4 | 4.0 | 4.0 | |
| 6 | 151 | 0 | 1.5 | 2.5 | 0 |
| | 151 | 2 | 1.0 | 2.0 | |
| 7 | 253 | 0 | 2.5 | 2.5 | 1 |
| | 253 | 4 | 5.0 | 5.0 | |
| 7 | 141 | 0 | 2.0 | 3.0 | 0 |
| | 141 | 2 | 3.0 | 3.5 | |
| 8 | 253 | 0 | 3.5 | 3.5 | 1 |
| | 253 | 2 | 5.5 | 5.5 | |
| 9 | 353 | 0 | 2.0 | 1.0 | 0 |
| | 353 | 2 | 2.5 | 1.5 | |

*First two digits indicate tooth number acording to the FDI World Dental Federation two-digit notation; third digid indicate site position: 1—mesio-buccal, 3—disto-buccal.
PD—pocket depth,
CAL—clinical attachment loss.

### Sample Collection

After removal of supragingival plaque, subgingival plaque samples were taken separately from the mesio-buccal and disto-buccal sites of pre-molars and first and second molars using individual sterile Gracey curettes and each sample placed in individual tubes containing 200 ul of RNAse-free buffer, immediately frozen and stored at −80° C. until processed.

### Community DNA and RNA Extraction

Cells were collected by centrifugation for 10 minutes at maximum speed in a microcentrifuge. 600 μL of MIRVANA™ kit lysis/binding buffer and 300 μl of 0.1-mm zirconia-silica beads (BIOSPEC© Products, Bartlesville, Okla.) were added to the samples. The beads were cleaned and sterilized beforehand with a series of HCl acid and bleach washes. Finally the beads were treated with Diethylpyrocarbonate (DEPC) overnight and autoclaved. Samples were bead-beated for 1 min at maximum speed. DNA and RNA were extracted simultaneously following the protocol of MIRVANA™ Isolation kit for RNA and TOTALLY RNA™ kit (Life Technologies) for DNA. Eukaryotic DNA was removed using the MOLYSIS® kit (Molzym GmbH & Co. KG, Bremen, Germany). MICROBENRICH™ (Life Technologies, the MICROBENRICH™ Kit employs hybridization capture technology to remove >90% of the rmRNA and rRNA from complex RNA populations) was used to remove eukaryotic RNA and MICROBEXPRESS™ to remove prokaryotic rRNA. All kits were used following manufacturer's instructions.

### DNA, RNA Amplification and Illumina Sequencing

DNA amplification was performed using the ILLUSTRA™ GENOMIPHI™ V2 amplification kit (GE Healthcare Life Sciences) according to manufacturer's instructions. RNA amplification was performed on total bacterial RNA using MESSAGE-AMP™ II-Bacteria RNA amplification kit (Applied Biosystems) following the manufacturer's instructions. Sequencing was performed at the Forsyth Institute. Illumina adapter-specific primers were used to amplify and selectively enrich for the cDNA generated from enriched mRNA. Quantified libraries were pooled and sequenced using the MISEQ™ v2, 2×150 cycle cartridge (Illumina). The NEXTERA™ XT kit was used to generate libraries from amplified DNA. Normalized libraries were pooled and sequenced using the 2×250 MISEQ™ v2 cartridge.

### Selection of Genomes in Databases

Genomes of archaea and bacteria and their associated information were downloaded from the Human Oral Microbiome Database (HOMD) server, the Pathosystems Resource Integration Center (PATRIC is the Bacterial Bioinformatics Resource Center, an information system designed to support the biomedical research community's work on bacterial infectious diseases via integration of vital pathogen information with rich data and analysis tools) ftp server (Wattam et al., Nucleic Acids Res.; 42:D581-591, 2014) and the J. Craig Venter Institute, a multidisciplinary genomic-focused organization. A total of 524 genomes from 312 species of bacteria and 2 genomes from 1 archaea species were used in the analysis (Table 5). Viral genomes were downloaded from NCBI's website (for genomes and viruses).

### Short Reads Sequence Alignment Analysis

Low-quality sequences were removed from the query files. Fast clipper and FASTQ quality filter from the FASTX-toolkit (a collection of command line tools for Short-Reads FASTA/FASTQ files preprocessing) were used to remove short sequences with quality score >20 in >80% of the sequence A GPS Pathfinder Office Geoid Grid File (gff) file was generated to map hits to different regions in the genomes of the database. Read counts from the SAM files were obtained using BEDTOOL MULTICOV™ from BEDTOOLS™ (Quinlan et al., Bioinforma Oxf Engl.; 26:841-2, 2010). BEDTOOL MULTICOV™ reports the count of alignments from multiple position-sorted and indexed BAM files (a binary version of SAM files) that overlap intervals in a BED file (a tab-delimited text file that defines a feature track).

### Phylogenetic Analysis of the Metagenome and Metatranscnptome

Counts from the DNA and RNA libraries were used to determine the phylogenetic composition of the respective libraries. A .gff file was created containing information on whole genomes that was used to assign hits to genomes. Abundance estimation at the species level was performed applying the Genome Abundance Similarity Correction (GASiC) proposed by Lindner and Renard to estimate true genome abundances via read alignment by considering reference genome similarities in a non-negative LASSO (least about shrinkage and selection operator) approach (an approach for predictions and estimations in high-dimensional linear models; Lindner et al., Nucleic Acids Res.; 41:e10, 2013). Estimated counts were normalized by frequency and log 2 transformed before final analysis. To identify significant differences between communities under the different conditions studied linear discriminant analysis (LDA) effect size (LefSe) was performed as proposed by Segata et al. (Genome Biol.; 12:R60, 2011) with default settings.

### Differential Expression Analysis

For assessing differential expression in genes within a specific species the transcript counts were normalized by the relative frequency of the species in the metagenome database. In the case of Gene Ontology (GO) and Kyoto Encyclopedia of Genes and Genomes (KEGG) pathways enrichment analysis they were not normalized by relative abundance since the whole community was treated as a single organism.

To identify differentially expressed genes from the RNA libraries, non-parametric tests were applied to the normalized counts using NOISEQ© BIO function of the R package NOISEQ© default conditions (k=0.5, 1c=1, replicates=“biological”) and Reads Per Kilobase per Million mapped reads (rpkm) normalization (rpkm option) using the threshold value for significance indicated by the authors of q=0.95 which for the function NOISEQ© BIO is equivalent to an false discovery rate (FDR) cutoff of 0.05 (Soneson et al., BMC Bioinformatics.; 14:91, 2013.; Tarazona et al., Genome Res.; 21:2213-23, 2011; the R. BIOCONDUCTOR© package NOISEQ©, is used for analyzing count data coming from next generation sequencing technologies).

### Gene Ontology (GO) Enrichment Analysis

To evaluate functional activities differentially represented in health or disease, the differentially expressed genes were mapped to known biological ontologies based on the Gene Ontology (GO) project (a collaborative effort to address the need for consistent descriptions of gene products across databases).

Enrichment analysis on these sets was performed using the R package GOseq (Gene Ontology analyzer for RNA-seq and other length biased data), which accounts for biases due to over-detection of long and highly expressed transcripts (Young et al., Genome Biol.; 11:R14, 2010). Gene sets with ≤10 genes were excluded from analysis. The reduce and visual Gene Ontology (REVIGO) web page (Kadowaki et al., J Biochem (Tokyo).; 128:153-9, 2000) was used to summarize and remove redundant GO terms from the results. Only GO terms with a false discovery rate (FDR)<0.05 were used. REVIGO plots were obtained for biological process categories:

### Quantification of Putative Virulence Factors

To identify putative virulence factors the Virulence Factors of Pathogenic Bacteria Database (VFDB) was used. A similar approach, but with less stringent conditions, has been used by other authors to identify putative virulence factors in genomic islands (Ho et al., PloS One.; 4:e8094, 2009). The VFDB contains 1205 virulence factors and 5955 virulence factors related genes from 75 pathogenic bacterial genera (Chen et al., Nucleic Acids Res.; 40:D641-645, 2012). A BLAST® similarity search of encoded proteins from the genomes in the applicants′ database was performed against the VFDB, with an e-value cutoff of 10⁻²⁵ and identity >99% to exclude distant homologs.

Integration of Metatranscnptomic Results with Clinical Traits

To integrate ‘omics’ results with clinical parameters the R package mixOmics (R package mixOmics provides statistical integrative techniques and variants to analyze highly dimensional data sets) was used (Gonzalez et al., BioData Min.; 5:19, 2012; Liquit et al., BMC Bioinformatics.; 13:325, 2012). The sparse Partial Least Square (sPLS) correlations between the clinical traits and species frequencies and profiles of gene expression in the periodontitis progressing sites were calculated. Metatranscriptome hits were normalized by frequencies obtained in the metagenome before ‘mixOmics’ analysis. For gene expression profiles low count genes were filtered using the NOISEQ© function filtered. data with filter 1 and a minimum count per million of 30 (Tarazona et al., Genome Res.; 21:2213-23, 2011). Correlation Circle plots were obtained on the sparse Partial Least Square (sPLS) results to visualize associations between principal components species and gene expression profiles. Relevance Networks showing correlations between genes and clinical traits were visualized in CYTOSCAPE© (an open source software platform for visualizing complex networks and integrating these with any type of attribute data) (Smoot et al., Bioinforma Oxf Engl.; 27:431-2, 2011) with a threshold correlation of 0.95.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

TABLE 5
Sequence ID Number listings with matching Gene/Gene Product Name

| | | Gene/Gene |
|---|---|---|
| SEQ ID NO | Organism | Product Name |
| SEQ ID NO. 1 | Porphyromonas gingivalis | CbiA |
| SEQ ID NO. 2 | Porphyromonas gingivalis | cbiA |
| SEQ ID NO. 3 | Porphyromonas gingivalis | CbiB |
| SEQ ID NO. 4 | Porphyromonas gingivalis | cbiB |
| SEQ ID NO. 5 | Porphyromonas gingivalis | CbiC |
| SEQ ID NO. 6 | Porphyromonas gingivalis | cbiC |
| SEQ ID NO. 7 | Porphyromonas gingivalis | CbiET |
| SEQ ID NO. 8 | Porphyromonas gingivalis | cbiET |
| SEQ ID NO. 9 | Porphyromonas gingivalis | CbiGF |
| SEQ ID NO. 10 | Porphyromonas gingivalis | cbiGF |
| SEQ ID NO. 11 | Porphyromonas gingivalis | CbiH |
| SEQ ID NO. 12 | Porphyromonas gingivalis | cbiH |
| SEQ ID NO. 13 | Porphyromonas gingivalis | CbiJD |
| SEQ ID NO. 14 | Porphyromonas gingivalis | cbiJD |
| SEQ ID NO. 15 | Porphyromonas gingivalis | CbiK |
| SEQ ID NO. 16 | Porphyromonas gingivalis | cbiK |
| SEQ ID NO. 17 | Porphyromonas gingivalis | CbiL |
| SEQ ID NO. 18 | Porphyromonas gingivalis | cbiL |
| SEQ ID NO. 19 | Porphyromonas gingivalis | CbiP |
| SEQ ID NO. 20 | Porphyromonas gingivalis | cbiP |
| SEQ ID NO. 21 | Porphyromonas gingivalis | CobA |
| SEQ ID NO. 22 | Porphyromonas gingivalis | cobA |
| SEQ ID NO. 23 | Porphyromonas gingivalis | CobD |
| SEQ ID NO. 24 | Porphyromonas gingivalis | cobD |
| SEQ ID NO. 25 | Porphyromonas gingivalis | CobS |
| SEQ ID NO. 26 | Porphyromonas gingivalis | cobS |
| SEQ ID NO. 27 | Porphyromonas gingivalis | CobT |
| SEQ ID NO. 28 | Porphyromonas gingivalis | cobT |
| SEQ ID NO. 29 | Porphyromonas gingivalis | CobU |
| SEQ ID NO. 30 | Porphyromonas gingivalis | cobU |
| SEQ ID NO. 31 | Porphyromonas gingivalis | HemD |
| SEQ ID NO. 32 | Porphyromonas gingivalis | hemD |
| SEQ ID NO. 33 | Tannerella forsythia | cbiE |
| SEQ ID NO. 34 | Tannerella forsythia | CbiE |
| SEQ ID NO. 35 | Tannerella forsythia | cbiD |
| SEQ ID NO. 36 | Tannerella forsythia | CbiD |
| SEQ ID NO. 37 | Tannerella forsythia | cobM |
| SEQ ID NO. 38 | Tannerella forsythia | CobM |
| SEQ ID NO. 39 | Tannerella forsythia | cobJ |
| SEQ ID NO. 40 | Tannerella forsythia | CobJ |
| SEQ ID NO. 41 | Tannerella forsythia | cobA |
| SEQ ID NO. 42 | Tannerella forsythia | CobA |
| SEQ ID NO. 43 | Tannerella forsythia | cobB |
| SEQ ID NO. 44 | Tannerella forsythia | CobB |
| SEQ ID NO. 45 | Tannerella forsythia | cobU |
| SEQ ID NO. 46 | Tannerella forsythia | CobU |
| SEQ ID NO. 47 | Tannerella forsythia | cobC |
| SEQ ID NO. 48 | Tannerella forsythia | CobC |
| SEQ ID NO. 49 | Tannerella forsythia | cobS |
| SEQ ID NO. 50 | Tannerella forsythia | CobS |
| SEQ ID NO. 51 | Tannerella forsythia | cobT |
| SEQ ID NO. 52 | Tannerella forsythia | CobT |
| SEQ ID NO. 53 | Tannerella forsythia | cobA2 |
| SEQ ID NO. 54 | Tannerella forsythia | CobA2 |
| SEQ ID NO. 55 | Tannerella forsythia | cbiL |
| SEQ ID NO. 56 | Tannerella forsythia | CbiL |
| SEQ ID NO. 57 | Tannerella forsythia | cobD |
| SEQ ID NO. 58 | Tannerella forsythia | CobD |
| SEQ ID NO. 59 | Tannerella forsythia | cobQ |
| SEQ ID NO. 60 | Tannerella forsythia | CobQ |
| SEQ ID NO. 61 | Tannerella forsythia | cbiK |
| SEQ ID NO. 62 | Tannerella forsythia | CbiK |
| SEQ ID NO. 63 | Tannerella forsythia | hemD |
| SEQ ID NO. 64 | Tannerella forsythia | HemD |
| SEQ ID NO. 65 | Fusobacterium nucleatum subspecies | cobA |
| | nucleatum | |
| SEQ ID NO. 66 | Fusobacterium nucleatum subspecies | CobA |
| | nucleatum | |
| SEQ ID NO. 67 | Fusobacterium nucleatum subspecies | cbiP |
| | nucleatum | |
| SEQ ID NO. 68 | Fusobacterium nucleatum subspecies | CbiP |
| | nucleatum | |
| SEQ ID NO. 69 | Fusobacterium nucleatum subspecies | cbiB |
| | nucleatum | |
| SEQ ID NO. 70 | Fusobacterium nucleatum subspecies | CbiB |
| | nucleatum | |
| SEQ ID NO. 71 | Fusobacterium nucleatum subspecies | cobS |
| | nucleatum | |
| SEQ ID NO. 72 | Fusobacterium nucleatum subspecies | CobS |
| | nucleatum | |
| SEQ ID NO. 73 | Fusobacterium nucleatum subspecies | cobU |
| | nucleatum | |
| SEQ ID NO. 74 | Fusobacterium nucleatum subspecies | CobU |
| | nucleatum | |
| SEQ ID NO. 75 | Fusobacterium nucleatum subspecies | cbiJ |
| | nucleatum | |
| SEQ ID NO. 76 | Fusobacterium nucleatum subspecies | CbiJ |
| | nucleatum | |
| SEQ ID NO. 77 | Fusobacterium nucleatum subspecies | cbiH |
| | nucleatum | |
| SEQ ID NO. 78 | Fusobacterium nucleatum subspecies | CbiH |
| | nucleatum | |
| SEQ ID NO. 79 | Fusobacterium nucleatum subspecies | cbiG |
| | nucleatum | |
| SEQ ID NO. 80 | Fusobacterium nucleatum subspecies | CbiG |
| | nucleatum | |
| SEQ ID NO. 81 | Fusobacterium nucleatum subspecies | cbiF |
| | nucleatum | |
| SEQ ID NO. 82 | Fusobacterium nucleatum subspecies | CbiF |
| | nucleatum | |
| SEQ ID NO. 83 | Fusobacterium nucleatum subspecies | cbiL |
| | nucleatum | |
| SEQ ID NO. 84 | Fusobacterium nucleatum subspecies | CbiL |
| | nucleatum | |
| SEQ ID NO. 85 | Fusobacterium nucleatum subspecies | cbiET |
| | nucleatum | |
| SEQ ID NO. 86 | Fusobacterium nucleatum subspecies | CbiET |
| | nucleatum | |
| SEQ ID NO. 87 | Fusobacterium nucleatum subspecies | cbiD |
| | nucleatum | |
| SEQ ID NO. 88 | Fusobacterium nucleatum subspecies | CbiD |
| | nucleatum | |
| SEQ ID NO. 89 | Fusobacterium nucleatum subspecies | cbiC |
| | nucleatum | |
| SEQ ID NO. 90 | Fusobacterium nucleatum subspecies | CbiC |
| | nucleatum | |
| SEQ ID NO. 91 | Fusobacterium nucleatum subspecies | cbiA |
| | nucleatum | |
| SEQ ID NO. 92 | Fusobacterium nucleatum subspecies | CbiA |
| | nucleatum | |
| SEQ ID NO. 93 | Fusobacterium nucleatum subspecies | cbiA2 |
| | nucleatum | |
| SEQ ID NO. 94 | Fusobacterium nucleatum subspecies | CbiA2 |
| | nucleatum | |
| SEQ ID NO. 95 | Fusobacterium nucleatum subspecies | cbiK |
| | nucleatum | |
| SEQ ID NO. 96 | Fusobacterium nucleatum subspecies | CbiK |
| | nucleatum | |
| SEQ ID NO. 97 | Fusobacterium nucleatum subspecies | cobA2 |
| | nucleatum | |
| SEQ ID NO. 98 | Fusobacterium nucleatum subspecies | CobA2 |
| | nucleatum | |
| SEQ ID NO. 99 | Prevotella intermedia | cobA |
| SEQ ID NO. | Prevotella intermedia | CobA |
| 100 | | |
| SEQ ID NO. | Eubacterium nodatum | cobH |
| 101 | | |
| SEQ ID NO. | Eubacterium nodatum | CobH |
| 102 | | |
| SEQ ID NO. | Eubacterium nodatum | cobQ |
| 103 | | |
| SEQ ID NO. | Eubacterium nodatum | CobQ |
| 104 | | |
| SEQ ID NO. 105 | Eubacterium nodatum | cbiK |
| SEQ ID NO. 106 | Eubacterium nodatum | CbiK |
| SEQ ID NO. 107 | Eubacterium nodatum | cobI |
| SEQ ID NO. 108 | Eubacterium nodatum | CobI |
| SEQ ID NO. 109 | Eubacterium nodatum | cobA |
| SEQ ID NO. 110 | Eubacterium nodatum | CobA |
| SEQ ID NO. 111 | Eubacterium nodatum | cobT |
| SEQ ID NO. 112 | Eubacterium nodatum | CobT |
| SEQ ID NO. 113 | Eubacterium nodatum | cobU |
| SEQ ID NO. 114 | Eubacterium nodatum | CobU |
| SEQ ID NO. 115 | Eubacterium nodatum | cobS |
| SEQ ID NO. 116 | Eubacterium nodatum | CobS |
| SEQ ID NO. 117 | Eubacterium nodatum | cbiD |
| SEQ ID NO. 118 | Eubacterium nodatum | CbiD |
| SEQ ID NO. 119 | Eubacterium nodatum | cobM |
| SEQ ID NO. 120 | Eubacterium nodatum | CobM |
| SEQ ID NO. 121 | Eubacterium nodatum | cbiG |
| SEQ ID NO. 122 | Eubacterium nodatum | CbiG |
| SEQ ID NO. 123 | Eubacterium nodatum | cobJ |
| SEQ ID NO. 124 | Eubacterium nodatum | CobJ |
| SEQ ID NO. 125 | Eubacterium nodatum | cobK |
| SEQ ID NO. 126 | Eubacterium nodatum | CobK |
| SEQ ID NO. 127 | Eubacterium nodatum | cbiET |
| SEQ ID NO. 128 | Eubacterium nodatum | CbiET |
| SEQ ID NO. 129 | Eubacterium nodatum | cbiA |
| SEQ ID NO. 130 | Eubacterium nodatum | CbiA |
| SEQ ID NO. 131 | Eubacterium nodatum | cbiA2 |
| SEQ ID NO. 132 | Eubacterium nodatum | CbiA2 |
| SEQ ID NO. 133 | Eubacterium nodatum | cobD |
| SEQ ID NO. 134 | Eubacterium nodatum | CobD |
| SEQ ID NO. 135 | Eubacterium nodatum | cbiK2 |
| SEQ ID NO. 136 | Eubacterium nodatum | CbiK2 |
| SEQ ID NO. 137 | Eubacterium nodatum | cysG |
| SEQ ID NO. 138 | Eubacterium nodatum | CysG |
| SEQ ID NO. 139 | Prevotella intermedia | cobA2 |
| SEQ ID NO. 140 | Prevotella intermedia | CobA2 |
| SEQ ID NO. 141 | Prevotella nigrescens | cobO |
| SEQ ID NO. 142 | Prevotella nigrescens | CobO |

What is claimed is:

1. An oral formulation comprising a cobalamin synthesis inhibitor to treat or prevent periodontal disease and related disorders.

2. The oral formulation of claim 1, wherein the cobalamin synthesis inhibitor is selected from the group consisting of 19-bromo-1-hydroxymethylbilane, N(D)-methyl-1-formylbilane, N-ethylmaleimide, adenylyl-imidodiphosphate, adenylyl(b,g-methylene)-diphosphonate, ADP, protonpump inhibitor (PPᵢ), divalent metal ions, S-adenosyl-L-homocysteine, tripolyphosphate/sodium triphosphate, and hydrogenobyrinic acid a,c-diamide.

3. The oral formulation of claim 1, wherein the cobalamin synthesis inhibitor reduces the level, activity, or expression of one or more cobalamin synthesis nucleic acids or polypeptides.

4. The oral formulation of claim 3, wherein the one or more polypeptides is selected from the group consisting of Delta-aminolevulinic acid dehydratase, Porphobilinogen deaminase, Uroporphyrinogen II synthase, Siroheme synthase, Precorrin-2 C20-methyltransferase, Precorrin-3B synthase, Precorrin-3B C17-methyltransferase, Precorrin-4 C11-methyltransferase, Precorrin-6A synthase, Precorrin-6X reductase, Precorrin-6Y C5,15-methyltransferase, Precorrin-8X methylmutase, Cobyrinic acid a,c-diamide synthase, Cobaltochelatase, Adenosylcobinamide-GDP ribazoletransferase, Nicotinate-nucleotide-dimethylbenzimidazole phosphoribosyltransferase, Cob(I)yrinic acid a,c-diamide adenosyltransferase, Cobyric acid synthase, Threonine-phosphate decarboxylase, Threonine-phosphate decarboxylase, Adenosylcobinamide kinase/Adenosylcobinamide-phosphate guanylyltransferase, Cobalamin-5-phosphate synthase, and Adenosylcobinamide kinase/Adenosylcobinamide-phosphate guanylyltransferase.

5. The oral formulation of claim 3, wherein the cobalamin synthesis inhibitor is one or more of an inhibitory nucleic acid or siRNA.

6. The oral formulation of claim 1, which comprises a toothpaste, powder, liquid dentifrice, mouthwash, subgingival irrigation fluid, mouth spray, mouth rinse, topically applied solution, denture cleanser, mouth guard, chewable tablets, chewing gum, lozenge, paste, gel, ointment, mucoadhesive, bioerodable film, buccal wafers, chocolate pieces, bars or nougats or candy.

7. The oral formulation of claim 1, which comprises a coated fiber.

8-10. (canceled)

11. A method for identifying a subject having or at risk of developing periodontal disease, the method comprising detecting altered expression of a gene associated with cobalamin synthesis, urea metabolism, citrate transport, iron ion transport, potassium ion transport, amino-acid transport, isoprenoid biosynthesis and ciliary and flagellar motility in a bacteria associated with periodontal disease, relative to a reference.

12. The method of claim 11, wherein the expression of a gene associated with cobalamin synthesis is increased, relative to a reference.

13. The method of claim 11, wherein the expression of a gene associated with potassium ion transport is decreased, relative to a reference.

14. A method for identifying a subject having or at risk of developing periodontal disease, the method comprising detecting an increase in expression of a polypeptide or gene encoding a polypeptide associated with cobalamin synthesis or a decrease in expression of a polypeptide or gene encoding a polypeptide involved in potassium ion transport in a bacteria associated with periodontal disease relative to a reference.

15. The method of claim 14, wherein the gene is identified at Table 5.

16. A method of treating or preventing periodontal disease by administering an oral formulation comprising a cobalamin synthesis inhibitor in a subject identified by an increase in expression of a polypeptide or gene involved in cobalamin synthesis or a decrease in expression of a polypeptide or gene encoding a polypeptide involved in potassium ion transport in a bacteria associated with periodontal disease relative to a reference.

17. The method of claim 16, wherein the cobalamin synthesis inhibitor is selected from the group consisting of 19-bromo-1-hydroxymethylbilane, N(D)-methyl-1-formylbilane, N-ethylmaleimide, adenylyl-imidodiphosphate, adenylyl(b,g-methylene)-diphosphonate, ADP, protonpump inhibitor (PPᵢ) divalent metal ions, S-adenosyl-L-homocysteine, tripolyphosphate/sodium triphosphate, and hydrogenobyrinic acid a,c-diamide.

18. The method of claim 16, wherein the cobalamin synthesis inhibitor reduces the level, activity, or expression of one or more cobalamin synthesis nucleic acids or polypeptides.

19. The method of claim 18, wherein the one or more polypeptides is selected from the group consisting of Delta-aminolevulinic acid dehydratase, Porphobilinogen deaminase, Uroporphyrinogen II synthase, Siroheme synthase, Precorrin-2 C20-methyltransferase, Precorrin-3B synthase, Precorrin-3B C17-methyltransferase, Precorrin-4 C11-methyltransferase, Precorrin-6A synthase, Precorrin-6X reductase, Precorrin-6Y C5,15-methyltransferase, Precorrin-8X methylmutase, Cobyrinic acid a,c-diamide synthase, Cobaltochelatase, Adenosylcobinamide-GDP ribazoletransferase, Nicotinate-nucleotide-dimethylbenzimidazole phosphoribosyltransferase, Cob(I)yrinic acid a,c-diamide adenosyltransferase, Cobyric acid synthase, Threonine-phosphate decarboxylase, Threonine-phosphate decarboxylase, Adenosylcobinamide kinase/Adenosylcobinamide-phosphate guanylyltransferase, Cobalamin-5-phosphate synthase, and Adenosylcobinamide kinase/Adenosylcobinamide-phosphate guanylyltransferase.

20. The method of claim 16, wherein the bacteria is Prevotella nigrescens, Prevotella intermedia, Fusobacterium nucleatum subspecies nucleatum, Tannerella forsythia, or Porphyromonas gingivalis.

21. A kit for detecting periodontitis in a subject, the kit comprising a panel of capture molecules that detect an alteration in the level of a polypeptide or gene encoding a polypeptide associated with cobalamin synthesis and/or potassium ion transport.

22. A kit for treating or preventing periodontitis, the kit comprising an effective amount of a cobalamin synthesis inhibitor.
